(19) United States
(12) Patent Application Publication
(10) Pub. No.: US 2025/0043240 A1
(43) Pub. Date: Feb. 6, 2025

(54) HYDROLYZED PROTEIN SERUM REPLACEMENT COMPOSITIONS

(71) Applicant: VIRGINIA TECH INTELLECTUAL PROPERTIES, INC., Blacksburg, VA (US)

(72) Inventors: Reza Ovissipour, Blacksburg, VA (US); Alexandrea Duscher, Blacksburg, VA (US); Inayat Batish, Blacksburg, VA (US); Mohammad Zarei, Blacksburg, VA (US)

(21) Appl. No.: 18/709,370

(22) PCT Filed: Nov. 10, 2022
(86) PCT No.: PCT/US2022/049589

§ 371 Date: May 10, 2024

Related U.S. Application Data
(60) Provisional application No. 63/278,839, filed on Nov. 12, 2021.

(51) Int. Cl. C12N 5/00; C07K 14/39; C07K 14/405; C07K 14/415; C07K 14/435; C12N 9/54

(52) U.S. Cl. CPC C12N 5/0018; C07K 14/39; C07K 14/405; C07K 14/415; C07K 14/435; C12N 9/54; C12Y 304/21014

(57) ABSTRACT

Described in several exemplary embodiments herein are serum replacement compositions, cell culture media containing said serum replacement compositions, and uses thereof.

### CROSS-REFERENCE TO RELATED APPLICATIONS

This application claims the benefit of and priority to U.S. Provisional Patent Application No. 63/278,839, filed on Nov. 12, 2021, the contents of which is incorporated by reference herein in its entirety.

### STATEMENT REGARDING FEDERALLY SPONSORED RESEARCH

This invention was made with government support under Grant No. 2021-69012-35978 awarded by NIFA. The government has certain rights in the invention.

### TECHNICAL FIELD

The subject matter disclosed herein is generally directed to cell culture media compositions, and more particularly serum replacement compositions for cell culture.

### BACKGROUND

The increase in the human population leads to many challenges, such as food shortages and hunger. The world population will reach 10 billion by 2050, requiring a 70% increase in meat production to meet global demand. Animal-based products, especially meats, are the primary food sources globally. It has been estimated that the requirement for animal-based meat will rise to 500 million tons to feed people by the year 2050. Among animal-based meat, seafood is an essential commodity that constitutes 20% of animal protein eaten, and its consumption has increased rapidly from previous years. Conventionally, seafood is obtained from the sea or cultivated in aquaculture. Capturing fish from the oceans is not sustainable, as it has already been exploited due to overfishing, fraud, by—catch, microplastic concerns, and pollution. Aquaculture has emerged as a viable alternative to capture fisheries, but it faces several challenges, including adding nutrients to the water, reliance on fisheries, antibiotics, emerging diseases, and permit issues. With all of these setbacks in the seafood industry, novel alternative meat sources are required to sustain the fisheries and aquaculture industry and environment in their current state. Many innovative meat substitutes have emerged, namely, insect-based proteins, cultivated meat, and plant-based protein alternatives. The problem with insect and plant-based alternatives is consumers' devotion to real meat, resulting in an unwillingness to consume non-authentic meat. In-vitro cultivated meat appears to be an excellent choice as it produces traditional meat with many environmental, economic, and health benefits over other alternatives. Cultured meat involves culturing cells or tissues in-vitro that promote its proliferation, metabolism, and growth. As such there exists a need for cell culture compositions and techniques suitable for use with animal cell culture, particularly for use with cultivating meat and other bioprodcuts.

Citation or identification of any document in this application is not an admission that such a document is available as prior art to the present invention.

### SUMMARY

Described in certain example embodiments herein are compositions comprising an amount of an algae protein hydrolysate, an amount of a plant protein hydrolysate, an amount of a fungi protein hydrolysate, an amount of an insect protein hydrolysate, an amount of a multicellular marine organism protein hydrolysate, or any combination thereof, wherein the composition is optionally a cell culture media supplement (e.g., a serum replacer), a partially complete cell culture media, or a complete cell culture media.

In certain example embodiments, the algae protein hydrolysate is prepared from: Chlorophyta (green algae), Rhodophyta (red algae), Stramenopiles (heterokonts), Xanthophyceae (yellow-green algae), Glaucocystophyceae (glaucocystophytes), Chlorarachniophyceae (chlorarachniophytes), (euglenids), Haptophyceae (coccolithophorids), Chrysophyceae (golden algae), Cryptophyta (cryptomonads), Dinophyceae (dinoflagellates), Haptophyceae (coccolithophorids), Bacillariophyta (diatoms), Eustigmatophyceae (eustigmatophytes), Raphidophyceae (raphidophytes), Scenedesmaceae and Phaeophyceae (brown algae), or any combination thereof. In some embodiments, the algal cell is selected from the group consisting of Chlamydomonas reinhardtii, Dunaliella salina, Haematococcus pluvialis, Chlorella vulgaris, Acutodesmus obliquus, Scenedesmus dimorphus, or any combination thereof. In certain example embodiments, the green alga is selected from the group consisting of Chlamydomonas, Dunaliella, Haematococcus, Chlorella, Scenedesmaceae, or any combination thereof. In some embodiments, the Chlamydomonas is a Chlamydomonas reinhardtii. In various embodiments, the Chlorella is a Chlorella minutissima or a Chlorella sorokiniana cell. Other algal cells of interest include without limitation, Gigartinaceae and Soliericeae of the class Rodophyceae (red seaweed): Chondrus crispus, Chondrus ocellatus, Eucheuma cottonii, Eucheuma spinosum, Gigartina acicularis, Gigartina pistillata, Gigartina radula, Gigartina stellate, Furcellaria fastigiata, Analipus japonicus, Eisenia bicyclis, Hizikia fusiforme, Kjellmaniella gyrata, Laminaria angustata, Laminaria longirruris, Laminaria Longissima, Laminaria ochotensis, Laminaria claustonia, Laminaria saccharina, Laminaria digitata, Laminaria japonica, Macrocystis pyrifera, Petalonia fascia, Scytosiphon lome, Gloiopeltis furcata, Porphyra crispata, Porhyra deutata, Porhyra perforata, Porhyra suborbiculata, Porphyra tenera, Rhodymenis palmate, or any combination thereof.

In certain example embodiments, wherein the plant protein hydrolysate is prepared from: Pea (Pisum sativum), Sorghum (Sorghum bicolor), rice (Oryza sativa), wheat (Triticum), Hemp (Cannabis sativa), quinoa (Chenopodium quinoa), soybean (Glycine max), Com, beans family, Chickpeas, Lentils, Peanuts, Almonds, Chia seeds, Potatoes, Seitan, oat, Almond, barley, Brewers Spent Grain, or any combination thereof.

In certain example embodiments, wherein the fungi protein hydrolysate is prepared from: yeasts including: Saccharomyces sp., Candida utilis, Lipomyces starkeyi and Phaffia rhodozyma, Fusarium moniliforme, Rhizopus niveus, Rhizopus oryzae, Aspergillus niger, Aspergillus oryzae, Candida guilliermondii, Candida lipolytica, Candida pseudotropicalis, Mucor pusillus Lindt, Mucor miehei, Rhizomucor miehei, Morteirella vinaceae, Endothia parasitica, Kluyveromyces lactis (previously called Saccharomyces lactis), Kluyveromyces marxianus, Lipomyces starkeyi, Rhodotorula colostri, Rhodotorula dairenensis, Rhodotorula glutinis, Rhodosporium diobovatum, Schizosaccharomyces pombe and Eremothecium ashbyii; Microfungi including Fusarium venenatum, mushrooms including Agaricus bisporus, Agaricus, Shiitake, Pleurotus ostreatus (oyster mushroom), Tremella fuciformis, Pleurotus eryngii, and or Enoki, orany combination thereof.

In certain example embodiments, wherein the insect protein hydrolysate is prepared from: Hermetia illucens, Galleria mellonella, Acheta domesticus, Dytiscus marginalis, Lethocerus indicus, Locusta migratoria, Gryllotalpa Gryllotalpa, Bombyx mori, Rhynchophorus ferrugineus, Blatta orientalis, Mesobuthus martensii, Gryllus bimaculatus, Acheta domesticus, Gryllus assimilis, Tenebrio molitor, Cicadidae, or any combination thereof.

In certain example embodiments, wherein the multicellular marine organism protein hydrolysate is prepared from: Arenicola marina, enchytraeus albidusm, Chironomidae, Hirudinea, Eisenia fetida, Perionyx excavates, Gastropoda, Mytilus edulis, Mytilus californianus, Crassostrea virginica, Crassostrea gigas, Stomolophus Meleagris, Lycastopsis catarractarum, Glycera, Nereidae, Artemia sp., Rotifera sp., Copepoda, Mercenaria mercenaria, Corbicula manilensis, Daphnia sp. Euphausiacea, Gammarus, or any combination thereof.

In certain example embodiments, the amount of each of the protein hydrolysates present in the composition ranges from any non-zero number greater than 0 to 100%, inclusive of all numerical values and all possible ranges therein.

In certain example embodiments, each hydrolysate present in the composition ranges in concentration (based on the total composition) any non-zero concentration greater than 0 mg/mL to 0.001 mg/mL, 0.01 mg/mL, 0.1 mg/mL, 1 mg/mL, 10 mg/mL, to/or 100 mg/mL, inclusive of all numerical values and all possible ranges therein.

In certain example embodiments, the average peptide size of the composition ranges from 2-50, 2-40, 2-30, 2-25, 2-20, 2-15, 2-10 or 2-5 amino acids, inclusive of all numerical values and all possible ranges therein.

In certain example embodiments, the degree of hydrolysis for any one of the hydrolysates ranges from 0 to 100%, inclusive of all numerical values and all possible ranges therein.

In certain example embodiments, the composition further comprises one or more nutrients (e.g., amino acids, vitamins, minerals, lipids, fatty acids, carbohydrates, sugars, and/or the like), one or more pH indicators, one or more antibiotics, one or more antifungals, one or more biologic factor or agent (e.g., enzymes, growth factors, attachment factors, hormones, immunomodulators, chemokines, cytokines, and/or the like), one or more buffering agents, one or more salt, one or more co-factor, one or more trace element, a cell viability colorimetric agent, and any combination thereof.

In certain example embodiments, the composition further comprises one or more cells.

In certain example embodiments, the one or more cells are eukaryotic cells.

Described in several example embodiments herein are methods of cell culture comprising culturing one or more cells in a composition of any one of the preceding paragraphs and as described elsewhere herein.

In certain example embodiments, culturing comprises, plating, growing, passaging, expanding, splitting, and/or any other conventional cell culture technique.

Described in several example embodiments herein are methods of preparing a composition in any one of any one of the preceding paragraphs and as described elsewhere herein, the method comprising: (a) blending one or more protein sources with water at a ratio of protein sources to water to form a slurry; (b) adding Alcalase to the slurry formed in (a) optionally at about 60 degrees C., optionally at a pH at about 6-8, optionally with agitation, for a period of time to form a hydrolysate and byproducts (e.g., oil and sludge); (c) inactivating enzymes present in the hydrolysate and byproducts; (d) separating the hydrolysate from the byproducts, optionally by centrifugation; and (e) optionally storing and/or retaining at least the hydrolysate.

In certain example embodiments, the one or more protein sources are selected from a plant, a fungi, an algae, an insect, a multicellular marine organism, or any combination thereof.

In certain example embodiments, the plant is Pea (Pisum sativum), Sorghum (Sorghum bicolor), rice (Oryza sativa), wheat (Triticum), Hemp (Cannabis sativa), quinoa (Chenopodium quinoa), soybean (Glycine max), Corn, beans family, Chickpeas, Lentils, Peanuts, Almonds, Chia seeds, Potatoes, Seitan, oat, Almond, barley, Brewers Spent Grain, or any combination thereof.

In certain example embodiments, the algae is Chlorophyta (green algae), Rhodophyta (red algae), Stramenopiles (heterokonts), Xanthophyceae (yellow-green algae), Glaucocystophyceae (glaucocystophytes), Chlorarachniophyceae (chlorarachniophytes), Euglenida (euglenids), Haptophyceae (coccolithophorids), Chrysophyceae (golden algae), Cryptophyta (cryptomonads), Dinophyceae (dinoflagellates), Haptophyceae (coccolithophorids), Bacillariophyta (diatoms), Eustigmatophyceae (eustigmatophytes), Raphidophyceae (raphidophytes), Scenedesmaceae and Phaeophyceae (brown algae), or any combination thereof. In some embodiments, the algal cell is selected from the group consisting of Chlamydomonas reinhardtii, Dunaliella salina, Haematococcus pluvialis, Chlorella vulgaris, Acutodesmus obliquus, Scenedesmus dimorphus, or any combination thereof. In some embodiments, the green alga is selected from the group consisting of Chlamydomonas, Dunaliella, Haematococcus, Chlorella, Scenedesmaceae, or any combination thereof. In some embodiments, the Chlamydomonas is a Chlamydomonas reinhardtii. In various embodiments the Chlorella is a Chlorella minutissima or a Chlorella sorokiniana cell. Other algal cells of interest include without limitation, Gigartinaceae and Soliericeae of the class Rodophyceae (red seaweed): Chondrus crispus, Chondrus ocellatus, Eucheuma cottonii, Eucheuma spinosum, Gigartina acicularis, Gigartina pistillata, Gigartina radula, Gigartina stellate, Furcellaria fastigiata, Analipus japonicus, Eisenia bicyclis, Hizikia fusiforme, Kjellmaniella gyrata, Laminaria angustata, Laminaria longirruris, Laminaria Longissima, Laminaria ochotensis, Laminaria claustonia, Laminaria saccharina, Laminaria digitata, Laminaria japonica, Macrocystis pyrifera, Petalonia fascia, Scytosiphon lome, Gloiopeltis furcata, Porphyra crispata, Porhyra deutata, Porhyra perforata, Porhyra suborbiculata, Porphyra tenera, and Rhodymenis palmate, or any combination thereof.

In certain example embodiments, the insect is Hermetia illucens, Galleria mellonella, Acheta domesticus, Dytiscus marginalis, Lethocerus indicus, Locusta migratoria, Gryllotalpa Gryllotalpa, Bombyx mori, Rhynchophorus ferrugineus, Blatta orientalis, Mesobuthus martensii, Gryllus bimaculatus, Acheta domesticus, Gryllus assimilis, Tenebrio molitor, Cicadidae, or any combination thereof.

In certain example embodiments, the multicellular marine organism is Arenicola marina, enchytraeus albidusm, Chironomidae, Hirudinea, Eisenia fetida, Perionyx excavates, Gastropoda, Mytilus edulis, Mytilus californianus, Crassostrea virginica, Crassostrea gigas, Stomolophus Meleagris, Lycastopsis catarractarum, Glycera, Nereidae, Artemia sp., Rotifera sp., Copepoda, Mercenaria mercenaria, Corbicula manilensis, Daphnia sp. Euphausiacea, Gammarus, or any combination thereof.

In certain example embodiments, the fungi is yeasts including: Saccharomyces sp., Candida utilis, Lipomyces starkeyi and Phaffia rhodozyma, Fusarium moniliforme, Rhizopus niveus, Rhizopus oryzae, Aspergillus niger, Aspergillus oryzae, Candida guilliermondii, Candida lipolytica, Candida pseudotropicalis, Mucor pusillus Lindt, Mucor miehei, Rhizomucor miehei, Morteirella vinaceae, Endothia parasitica, Kluyveromyces lactis (previously called Saccharomyces lactis), Kluyveromyces marxianus, Lipomyces starkeyi, Rhodotorula colostri, Rhodotorula dairenensis, Rhodotorula glutinis, Rhodosporium diobovatum, Schizosaccharomyces pombe and Eremothecium ashbyii; Microfungi including Fusarium venenatum, mushrooms including Agaricus bisporus, Agaricus, Shiitake, Pleurotus ostreatus (oyster mushroom), Tremella fuciformis, Pleurotus eryngii, Enoki, or any combination thereof.

Described in certain example embodiments herein are kits comprising the composition of any one of the paragraphs and as described elsewhere herein.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—An embodiment of a process of enzymatic hydrolysis for various raw materials for generating a composition of the present disclosure.

FIG. 2—Results of cell culture with yeast hydrolysate+media with 10% serum.

FIG. 3—Results of cell culture with yeast hydrolysate+media with 5% serum.

FIG. 4—Results of cell culture with yeast hydrolysate+media with 0% serum.

FIG. 5—Overall micrographs of cell cultures with yeast hydrolysates.

FIG. 6—Results of cell culture with black soldier fly (BSF) hydrolysate (BSFH)+media with 10% serum.

FIG. 7—Results of cell culture with BSF hydrolysate (BSFH)+media with 5% serum.

FIG. 8—Results of cell culture with BSF hydrolysate+media with 0% serum.

FIG. 9—Phase contrast microscopic images of cells cultured in various BSF hydrolysate amounts with and with serum.

FIG. 10—The effect of growth factors in media with 5% serum.

FIG. 11—The effect of growth factors in media with 0% serum.

FIG. 12A-12E-(FIG. 12A) Protein hydrolysates bioprocessing via enzymatic reaction; Techno-functional properties of protein hydrolysates; (FIG. 12B) oil holding capacity; (FIG. 12C) emulsifying capacity; (FIG. 12D) foaming capacity, and (FIG. 12E) degree of hydrolysis.

FIG. 13A-13C-Cell growth parameters of ZEM2S cells at various serum concentrations: (FIG. 13A): The effect of different concentrations of serum on cell morphology and density through phase contrast microscope; (FIG. 13B) Cell density at different concentrations of serum. ns: non-significant, (**) significant (P<0.05); (FIG. 13C) doubling time calculated based on the specific growth rate of the cells. Micrographs of ZEM2S cells were obtained at 10× (200 μm) magnification.

FIG. 14A-14B-ZEM2S cells growth in media containing different serum concentrations (10%, 5% and 0%), (FIG. 14A) 10 mg/mL protein hydrolysates (AI: cell growth; AII: cell viability) and (FIG. 14B) 1 mg/mL protein hydrolysate (BI: cell growth; BII: cell viability).

FIG. 15A-15C-ZEM2S cells growth in media containing different serum concentrations (0, 5, and 10%), (FIG. 15A) 0.1 mg/mL protein hydrolysates (AI) cell growth, (AII) cell viability; (FIG. 15B) 0.01 mg/mL protein hydrolysates, (B1) cell growth, (BII) cell viability; (FIG. 15C) 0.001 mg/mL protein hydrolysates, (C1) cell growth, (CII) cell viability.

FIG. 16-ZEM2S cells morphological changes in different media containing BSF hydrolysates (0-10 mg/mL) in combination with 0, 5, and 10% serum. The morphology was provided using phase contrast microscope (200 μm).

FIG. 17A-17C-ZEM2S cells growth in media containing different serum concentrations (0, 1, 2.5, and 10%), (FIG. 17A) 0.1 mg/mL protein hydrolysates (AI) cell growth, (AII) cell viability; (FIG. 17B) 0.01 mg/mL protein hydrolysates, (B1) cell growth, (BII) cell viability; (FIG. 17C) 0.001 mg/mL protein hydrolysates, (C1) cell growth, (CII) cell viability. For the x-axis in FIG. 17B-17D: (1)=0% serum, (2)=1% serum (3)=2.5% serum, (4)=5% serum, (5)=10% serum, (6)=0.001 mg/mL (1%), (7)=0.01 mg/mL (1%), (8)=0.1 mg/mL (1%), (9)=0.001 mg/mL (2.5%), (10)=0.01 mg/mL (2.5%), (11)=0.1 mg/mL (2.5%)

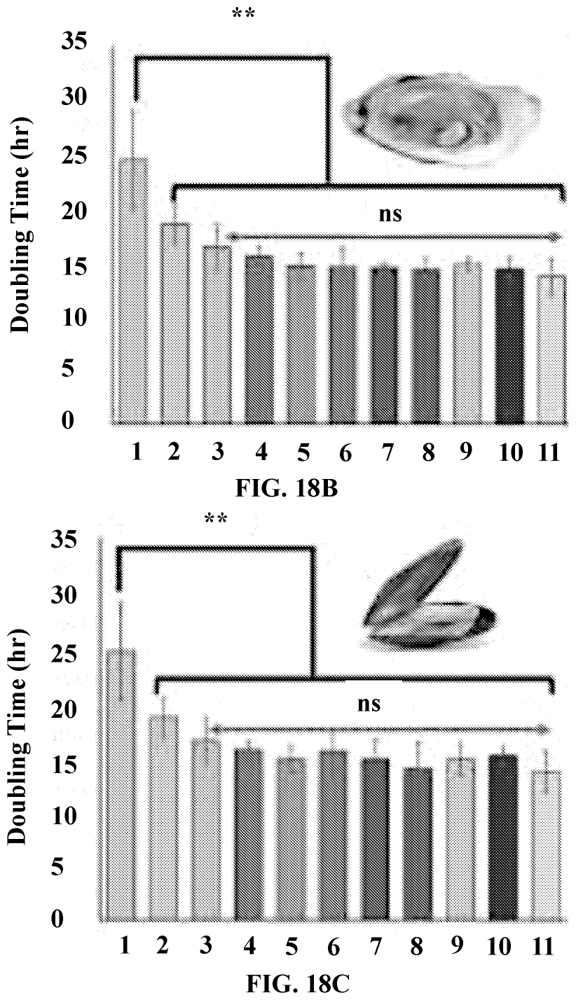

FIG. 18A-18E-(FIG. 18A) Hoechst and actin green, fluorescent staining of cells at all serum and protein hydrolysate conditions. Only selected concentrations of protein hydrolysates in combination with 1% serum were used for imaging; Doubling time (hr) for protein hydrolysates at low concentrations (0.001, 0.01, and 0.1 mg/mL) in combination with low serum concentrations (1, and 2.5%), negative control (serum-free) and positive control (10% serum), for oyster (FIG. 18B); mussel (FIG. 18C); lugworm (FIG. 18D); and BSF (FIG. 18E). ns: non-significant; (**) significant (P<0.05).

FIG. 19-Lactate dehydrogenase assay conducted for protein hydrolysate conditions at 2.5 and 1% serum concentrations. a represents significantly different (P<0.05) from 10% control, b represents significantly different (P<0.05) from 0% control.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R.I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

Definitions of common terms and techniques in chemistry and organic chemistry can be found in Smith. Organic Synthesis, published by Academic Press. 2016; Tinoco et al. Physical Chemistry, 5th edition (2013) published by Pearson; Brown et al., Chemistry, The Central Science 14th ed. (2017), published by Pearson, Clayden et al., Organic Chemistry, 2nd ed. 2012, published by Oxford University Press; Carey and Sunberg, Advanced Organic Chemistry, Part A: Structure and Mechanisms, 5th ed. 2008, published by Springer; Carey and Sunberg, Advanced Organic Chemistry, Part B: Reactions and Synthesis, 5th ed. 2010, published by Springer, and Vollhardt and Schore, Organic Chemistry, Structure and Function; 8th ed. (2018) published by W. H. Freeman.

Definitions of common terms and techniques in cell culture can be found in General Techniques of Cell Culture. Handbooks in Practical Animal Cell Biology. Harrison, M. A. and I. F. Rae. Cambridge University Press, 1997; Ed. Aschner et al., Cell Culture Techniques. Springer Protocol. Humana Press. 2011; and Capes-Davis, A. and R. I. Freshney. Freshney's Culture of Animal Cells: A manual of Basic Technique and Specialized Applications, 8th Edition.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The first step in producing cultivated meat is generating a source of stable, self-renewing cells that possess a high proliferation rate and differentiation potential. Cells that can be considered for this purpose are Embryonic Stem Cells (ESC's), induced Pluripotent Stem Cells (iPSC's), Adult Stem Cells (ASC's), satellite cells, and specific dedifferentiated cells. Unlike other cell lines and sources, ESC's have higher pluripotency and the capacity to differentiate, making them perfect starting cells for the cultivation of meat and forming a cell repository.

The second critical factor in cell culture technology is the formulation of culture media. A culture medium sustains the cell or tissue's proliferation and growth (Yao and Asayama Reprod Med Biol. 2017-3-21;16 (2): 99-1172017). Fetal Bovine Serum (FBS) is the most used media component that aids cell proliferation and metabolism. However, many factors discourage its use, such as high cost, ill-defined formulation, high demand-less supply, high variability, inability to grow specific cells, and ethical source issues (van der Valk et al. 2017. Alternatives to Laboratory Animals. 45 (6) https://doi.org/10.1177/02611929170450061). Cell culture media accounts for more than 99 percent of the total cost of the cultivated meat process (Humbird, D. Scale-up economics for cultured meat. Biotechnology and Bioengineering 118, 8 (2021) and Risner, D. et al. Preliminary techno-economic assessment of animal cell-based meat. Foods 10, 3 (2020)), developing a less expensive media would significantly reduce the cost of cultivated meat. According to Good Food Institute's study, over 95% of serum-free media contain growth factors and hormones, which contribute to the cost. Additionally, these factors and hormones are critical for cell proliferation and viability (An analysis of culture medium costs and production volumes for cultivated meat. L Specht, S Scientist—The Good Food Institute: Washington, DC, USA, 2020). Thus, developing a serum-free medium with less expensive sources of growth factors and hormones would be an efficient way to industrialize cultivated meat.

Embodiments disclosed herein can provide serum replacement compositions and cell culture media containing the serum replacement compositions and uses thereof. In some embodiments, the serum replacement compositions contain hydrolysate from unconventional sources, including but not limited to, plants, fungi, insects, multicellular marine organisms, algae, and combinations thereof. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Serum Replacement and Cell Culture Compostions

Described in several example embodiments herein are serum replacement compositions and cell culture media compositions. In some embodiments, the composition can be composed in part or in whole of an amount of an algae protein hydrolysate, an amount of a plant protein hydrolysate, an amount of a fungi protein hydrolysate, an amount of an insect protein hydrolysate, an amount of a multicellular marine organism protein hydrolysate, or any combination thereof. As used herein, “hydrolysate” refers to proteins or peptides that are chemically, enzymatically, or otherwise broken down to smaller polypeptides, peptides, and/or amino acids. Techniques and methods for preparing protein hydrolysates are described herein and are generally known in the art.

In some embodiments the composition is formulated as a serum replacement composition or cell culture media supplement. In this context, the composition is formulated to be added to a cell culture media. In some embodiments, the compositions is formulated as a partial cell culture media (or partially complete). In this context, the composition includes components that form the serum replacement composition and one or more other components that form a cell culture media but does not make a complete cell culture media. In some embodiments, the composition is formulated as a complete cell culture media. In this context, the complete cell culture media includes the serum replacement composition and other components that form a complete cell culture media. In some embodiments, the composition is a cell culture media supplement, a partially complete cell culture media, or a complete cell culture media. In some embodiments, the composition is a serum replacer. In some embodiments, the composition does not contain serum.

In some embodiments, the average peptide size in the composition ranges from 2-50 amino acids, such as 2, to/or 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids, including any individual value or any range of values therein. In some embodiments, the average peptide size of each protein hydrolysate in the composition independently ranges from 2-50 amino acids, such as 2, to/or 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids, including any individual value or any range of values therein. In certain example embodiments, the average peptide size of the composition ranges from 2-50, 2-40, 2-30, 2-25, 2-20, 2-15, 2-10 or 2-5 amino acids, inclusive of all numerical values and all possible ranges therein. In some embodiments, the average peptide size between any two different protein hydrolysates is the same. In some embodiments, the average peptide size between any two different protein hydrolysates is the different.

In some embodiments, the degree of hydrolysis for each protein hydrolysate in the composition independently ranges from 0 to 100%, such as 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, including any individual value or any range of values therein. In some embodiments, the degree of hydrolysis between any two different protein hydrolysates in the composition is the same. In some embodiments, the degree of hydrolysis between any two different protein hydrolysates in the composition is the different. In some embodiments, the degree of hydrolysis for any one or more of the hydrolysates independently ranges from 0 to 100%, such as 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, including any individual value or any range of values therein.

### Algae Protein Hydrolysates

Any suitable algae source can be used to prepare an algae protein hydrolysate. In some embodiments, the algae the algae protein hydrolysate is prepared from: Chlorophyta (green algae), Rhodophyta (red algae), Stramenopiles (heterokonts), Xanthophyceae (yellow-green algae), Glaucocystophyceae (glaucocystophytes), Chlorarachniophyceae (chlorarachniophytes), Euglenida (euglenids), Haptophyceae (coccolithophorids), Chrysophyceae (golden algae), Cryptophyta (cryptomonads), Dinophyceae (dinoflagellates), Haptophyceae (coccolithophorids), Bacillariophyta (diatoms), Eustigmatophyceae (eustigmatophytes), Raphidophyceae (raphidophytes), Scenedesmaceae and Phaeophyceae (brown algae), or any combination thereof. In some embodiments, the algae protein hydrolysate is prepared from Chlamydomonas reinhardtii, Dunaliella salina, Haematococcus pluvialis, Chlorella vulgaris, Acutodesmus obliquus, Scenedesmus dimorphus, and any combination thereof. In some embodiments, the algae protein hydrolysate is prepared from Chlamydomonas, Dunaliella, Haematococcus, Chlorella, Scenedesmaceae, and any combination thereof. In some embodiments, the algae protein hydrolysate is prepared from (a) the Chlamydomonas is a Chlamydomonas reinhardtii, (b) the Chlorella is a Chlorella minutissima or a Chlorella sorokiniana, or both (a) and (b).

In some embodiments, the algae protein hydrolysate is prepared from a Rodophyceae (red seaweed), optionally a Gigartinaceae or a Soliericeae. In some embodiments, the Rodophyceae is Chondrus crispus, Chondrus ocellatus, Eucheuma cottonii, Eucheuma spinosum, Gigartina acicularis, Gigartina pistillata, Gigartina radula, Gigartina stellate, Furcellaria fastigiata, Analipus japonicus, Eisenia bicyclis, Hizikia fusiforme, Kjellmaniella gyrata, Laminaria angustata, Laminaria longirruris, Laminaria Longissima, Laminaria ochotensis, Laminaria claustonia, Laminaria saccharina, Laminaria digitata, Laminaria japonica, Macrocystis pyrifera, Petalonia fascia, Scytosiphon lome, Gloiopeltis furcata, Porphyra crispata, Porhyra deutata, Porhyra perforata, Porhyra suborbiculata, Porphyra tenera, Rhodymenis palmate, or any combination thereof.

Other suitable algae include, without limitation, Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

### Plant Protein Hydrolysates

Any suitable plant source can be used to prepare an algae protein hydrolysate. In some embodiments, the plant protein hydrolysate is prepared from Pea (Pisum sativum), Sorghum (Sorghum bicolor), rice (Oryza sativa), wheat (Triticum), Hemp (Cannabis sativa), quinoa (Chenopodium quinoa), soybean (Glycine max), Corn, a plant of the bean family, Chickpeas, Lentils, Peanuts, Almonds, Chia seeds, Potatoes, Seitan, oat, Almond, barley, Brewers Spent Grain, or any combination thereof.

Other suitable plant protein sources include species of Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga, or any combination thereof. Further suitable plants include, without limitation, crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Additional suitable plants include, without limitation, angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, elementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini.

### Fungi Protein Hydrolysate

Any suitable fungi can be used to prepare the fungi protein hydrolysate. In some embodiments, the fungi protein hydrolysate is prepared from yeast. In some embodiments, the yeast is Saccharomyces sp., Candida utilis, Lipomyces starkeyi and Phaffia rhodozyma, Fusarium moniliforme, Rhizopus niveus, Rhizopus oryzae, Aspergillus niger, Aspergillus oryzae, Candida guilliermondii, Candida lipolytica, Candida pseudotropicalis, Mucor pusillus Lindt, Mucor miehei, Rhizomucor miehei, Morteirella vinaceae, Endothia parasitica, Kluyveromyces lactis (previously called Saccharomyces lactis), Kluyveromyces marxianus, Lipomyces starkeyi, Rhodotorula colostri, Rhodotorula dairenensis, Rhodotorula glutinis, Rhodosporium diobovatum, Schizosaccharomyces pombe, Eremothecium ashbyii; Microfungi, optionally Fusarium venenatum, mushrooms, optionally Agaricus bisporus, Agaricus, Shiitake, Pleurotus ostreatus (oyster mushroom), Tremella fuciformis, Pleurotus eryngii, and/or Enoki, or any combination thereof.

Other suitable fungi that can be used to prepare the fungi protein hydrolysate include without limitation, mushrooms, puffballs, stinkhorns, bracket fungi, jelly fungi, boletes, smuts, and bunts. In some embodiments, the fungi protein hydrolysate is formed from a fungus of the species Amanita, Aposphaeria, Armillaria, Ascochyta, Aspergillus, Boletus, Candida, Conocybe, Coprinopsis, Coprinus, Cortinarius, Cyathus, Entoloma, Fusarium, Gymnopilus, Gymnopus, Hebeloma, Hygrocybe, Hygrophorus, Inocybe, Lactarius, Lactifluus, Leccinum, Lepiota, Leucoagaricus, Leucocoprinus, lichen, Mortierella, Rinodina, Verrucaria, Marasmius, Mycosphaerella, Panaeolus, Penicillium, Peniophora, Phaeocollybia, Pholiota, Pholiota, Pholiotina, Pleurotus, Pluteus, Psathyrella, Psilocybe, Puccinia, Russula, Serpula, Trametes, Trichoderma, Tricholoma, Tulostoma, and combinations thereof.

### Insect Protein Hydrolysates

Any suitable insect can be used to prepare the insect protein hydrolysate. In some embodiments, the insect protein hydrolysate is prepared from Hermetia illucens, Galleria mellonella, Acheta domesticus, Dytiscus marginalis, Lethocerus indicus, Locusta migratoria, Gryllotalpa Gryllotalpa, Bombyx mori, Rhynchophorus ferrugineus, Blatta orientalis, Mesobuthus martensii, Gryllus bimaculatus, Acheta domesticus, Gryllus assimilis, Tenebrio molitor, Cicadidae, or any combination thereof.

### Multicellular Marine Organism Protein Hydrolysate

Any suitable multicellular marine organism can be used to prepare the multicellular marine organism protein hydrolysate. In some embodiments, the multicellular marine organism protein hydrolysate is prepared from Arenicola marina, enchytraeus albidusm, Chironomidae, Hirudinea, Eisenia fetida, Perionyx excavates, Gastropoda, Mytilus edulis, Mytilus californianus, Crassostrea virginica, Crassostrea gigas, Stomolophus Meleagris, Lycastopsis catarractarum, Glycera, Nereidae, Artemia sp., Rotifera sp., Copepoda, Mercenaria mercenaria, Corbicula manilensis, Daphnia sp. Euphausiacea, Gammarus, or any combination thereof.

### Amounts of Protein Hydrolysates

Each amount of a protein hydrolysate contained in the compositions of the present disclosure can be independently determined. In some embodiments, the amount of each of the protein hydrolysates present in the composition as a percent of the total composition or of the hydrolysate portion of the composition independently ranges from any non-zero number greater than 0 to 100% w/w, v/v, w/v, or wt. %, including any individual value or any range of values therein, such as 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% w/w, v/v, w/v, or wt. %, including any individual value or any range of values therein.

In some embodiments, the total amount of all protein hydrolysates in the composition as a percent of the total composition ranges from any non-zero number greater than 0 to 100% w/w, v/v, w/v, or wt. %, including any individual value or any range of values thereof, such as 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% w/w, v/v, w/v, or wt. %, including any individual value or any range of values therein.

In some embodiments, each hydrolysate present in the composition independently ranges in concentration, as based on the total composition, any non-zero concentration greater than 0 mg/mL to 100 mg/mL, such as 0.1 to/or 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mg/mL, including any individual value or any range of values therein.

### Exemplary Additional Components

The compositions of the present disclosure can further include one or more additional components suitable for cell culture. In certain example embodiments, the composition further comprises one or more nutrients (e.g., amino acids, vitamins, minerals, lipids, fatty acids, carbohydrates, sugars, and/or the like), one or more pH indicators, one or more antibiotics, one or more antifungals, one or more biologic factor or agent (e.g., enzymes, growth factors, attachment factors, hormones, immunomodulators, chemokines, cytokines, and/or the like), one or more buffering agents, one or more salt, one or more co-factor, one or more trace element, a cell viability colorimetric agent, and any combination thereof.

Exemplary hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g. arachidonic acid, lipoxins, and prostaglandins), and steroid hormones (e.g. estradiol, testosterone, tetrahydro testosterone, cortisol).

Exemplary immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g. IFN-α, IFN-β, IFN-ε, IFN-K, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Exemplary growth factors include, without limitation, EGF, FGF (e.g., FGF1-FGF23), NGF, PDGF, VEGF, IGF, CSF, M-CSF, G-CSF, GMCSF, GCSF, Erythropieitn, TPO, a BMP, HGF, GDF, Neurotrophins (e.g., BDNF, NGF, NT-3, NT-4), MSF, SGF, GDF,an adrenomedullin, an angiopoietin, an autocrine motility factor, CNTF,LIF, IL-6, an ephrin, EPO, FBS, GDNF, neurturin, persephin, artemin, GDF9, HGF, KGF, MSF, MSP, myostatin, a neruorgulin (e.g., NRG1-NRG4), PGF, fenalase, TCGF, TGF alpha, TGF beta, TNF alpha, and any combination thereof.

Exemplary pH indicators includes, without limitation, phenol red.

### Cells

In some embodiments, the composition can include one or more cells. In some embodiments, the cells are eukaryotic cells. In some embodiments, the cells are mammalian cells. In some embodiments, the cells are non-mammalian cells. In some embodiments, the cells are avian cells. In some embodiments, the cells are human cells. In some embodiments, the cells are non-human animal cells. In some embodiments, the cells are bovine, porcine, ovine, equine, canine, feline, cervine, murine, or any combination thereof. In some embodiments, the cells are in suspension within the composition. In some embodiments, the composition and the cells are contained within a vessel or other container. In some embodiments, the cells are adherent to one or more surfaces of the vessel or container.

### Methods of Preparing Serum Replacement Compositions

Described herein are methods of preparing a composition of the present disclosure. In some embodiments, the method includes blending one or more protein sources with water at a ratio of protein sources to water to form a slurry; adding alcalase to the slurry formed in (a) for a period of time to form a hydrolysate and one or more byproducts; inactivating enzymes present in the hydrolysate and the one or more byproducts; separating the hydrolysate from the one or more byproducts; and optionally storing and/or retaining at least the hydrolysate.

In some embodiments, (b) is performed at a temperature ranging from about 45 to about 65 degrees C., or any value or any range of values therein, such as 45 to/or 46, 47, 48, 49, 50, 51, 52, 53, 54, 55, 56, 57, 58, 59, 60, 61, 62, 63, 64, 65 degrees C. In some embodiments, (b) is performed at about 60 degrees C.

In some embodiments, (b) is performed at a pH of about 6-8, or any value or any range of values therein, such as 6, to/or 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8.

In some embodiments, (b) is performed with agitation. In some embodiments, (b) is performed without agitation.

In some embodiments, the one or more byproducts are oil, sludge, or both. In some embodiments, (d) separating comprises centrifuging the hydrolysate and the one or more byproducts.

The method of any one of aspects 24-29, wherein the one or more protein sources are selected from a plant, a fungi, an algae, an insect, a multicellular marine organism, or any combination thereof.

Suitable plants, algae, fungi, insects, and multicellular marine organisms for use in a method to prepare the compositions are previously described elsewhere herein at least in connection with the sources for protein hydrolysate. Such discussion is recapitulated in the present context.

### Cell Culture Methods

Also described herein are methods of cell culture using the compositions of the present disclosure. Any suitable cell can be cultured in the compositions of the present disclosure. In some embodiments, the cell culture is a cultivated meat or other food product cell culture. In some embodiments, the cell culture method does not include the use of serum.

Described in certain example embodiments herein, are methods of cell culture that include culturing one or more cells in a composition of the present invention. General cell culture techniques are discussed elsewhere herein. In some embodiments, culturing can include, plating cells, growing cells, passaging cells, expanding cells, splitting cells, imaging cells, refreshing cell culture media, collecting cells, and/or the like. Such techniques can occur one or more times. In some embodiments, the cells are collected and used or stored. Suitable cell storage techniques are generally known in the art.

### Kits and Devices

Also described herein kits, such as combination kits, that contain one or more compositions of the present disclosure. Any of the compounds, compositions, formulations, particles, cells, and/or devices described herein, or a combination thereof can be presented as a combination kit. As used herein, the terms “combination kit” or “kit of parts” refers to the compounds, compositions, formulations, particles, cells, devices and any additional components that are used to package, sell, market, deliver, use and/or administer the combination of elements or a single element, such as the active ingredient, contained therein. Such additional components include, but are not limited to, packaging, syringes, blister packages, bottles, and the like. When one or more of the compounds, compositions, formulations, particles, and/or cells, herein or a combination thereof contained in the kit are used simultaneously, the combination kit can contain the active agents in a single composition or formulation or in separate formulations or compositions. When the compounds, compositions, formulations, particles, and cells described herein or a combination thereof and/or kit components are not administered simultaneously, the combination kit can contain each agent or other component in separate formulations or compositions. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, etc. described herein or a combination thereof contained therein, safety information and/or content, origin, etc. regarding the content of the compounds, compositions, formulations particles, and/or cells described herein.

Also described herein, are devices that can include one or more compositions of the present disclosure and optionally one or more cells. In some embodiments, the device is a cell culture dish, plate, or other cell culture container or vessel. In some embodiments, the cells are contained in one or more wells of a cell culture dish, plate or other cell culture container or vessel. In some embodiments, the cells are in suspension. In some embodiments, the cells are adherent to a surface of the device.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

### Introduction

### Fetal Bovine Serum (FBS) and its Substitutions

Nutrient composition in cell culture media dramatically influences the cell's growth-supporting microenvironment. Standard basal media formulations include buffered solutions of salts, sugars, vitamins, and amino acids (e.g., Dulbecco's Modified Eagle's Medium, DMEM). Proteins and peptides, not found in basal media, are vital for cell proliferation because they serve as a source of building blocks, energy, and signaling molecules and they also play an essential role in media preparation (Sinacore et al. 2000; Freshney 2016). The most used media supplement for cell culture that contains these important proteins is Fetal Bovine Serum (FBS), also called serum. Serum is a clear biological fluid, a complex mixture of macromolecules derived from fetal calves' blood and is the most common source of proteins and peptides in media (Logarušić et al. 2021). The serum is an excellent media supplement that consists of various constituents with optimum concentrations that promote cell proliferation and development. Following is the list of ingredients that serum possesses: polypeptides and proteins, growth factors, amino acids, lipids, carbohydrates, polyamine, urea, inorganic moieties, hormones, and vitamins (Freshney 2015). Fetal bovine serum has a substantial production globally with approximately 500,000 liters harvested per year (Brunner et al. 2010). However, it has a fair share of disadvantages: ethically questionable extraction, high demand-low supply, poorly defined components, batch to batch variation, high probability of inherent contamination, and inability to grow many specialized cells (Galbraith et al. 2018). These challenges give rise to the need of fabricating a novel media, free of serum that can counter the disadvantages posed by serum. Serum-free and chemically defined media are available options for substituting serum (Kuo et al. 2020).

Serum-free media are devoid of serum and other unprocessed biological fluids of human or animal origin, but highly pure animal and human supplements can be added. Some examples are: animal and plant hydrolysates, plate lysates, serum fractions, purified recombinant proteins, etc., are utilized (Karnieli et al. 2017). However, serum-free media have shown inconsistencies in the growth of various cells and may contain components that are cannot be defined which impart variability (Bhat et al. 2021).

Chemically defined media (CDM) is a subset of serum-free media in which all of the constituents added are extremely pure and precisely defined at the molecular level (Freshney 2016). Even though chemically defined media (CDM) is preferred, it often inhibits cell growth compared to serum-containing media because different cells have complex and variable nutritional requirements. As a result, CDM fabrication is a time-consuming and challenging task, and its supplementation with hydrolysates is a good alternative (Ling et al. 2015).

Protein hydrolysates are defined as a composite blend of many bioactive entities that is composed of varying size of oligopeptides, peptides and amino acids and produced by complete and partial hydrolysis of different protein sources (Nasri 2017). Protein hydrolysates act as a source of fatty acids, namely oleic acid, linoleic and linolenic acids, phospholipids, carbohydrates, vitamins etc. Due to the presence of many bioactive compounds, peptides, proteins, and lipids, protein hydrolysates would construct an appropriate and efficacious substitute for serum. Some advantages of protein hydrolysates are: 1) Analogous to serum, the hydrolysate is also a concoction of many bioactive moieties like lipids, carbohydrates, vitamins etc. that augment the growth, survival, and proliferation of cells; 2) Protein hydrolysates economically and ethically are viable; 3) Hydrolysates have already been used and successfully implemented in animal cell culture.

Despite all the advantages offered by it, hydrolysate also has some disadvantages: 1) similar to serum, the composition is not clearly defined as well as may have batch to batch variability; 2) May also have high risk of contamination (Yao and Asayama 2017). Despite these issues, protein hydrolysates had been used by many researchers as growth media and serum substitutes for cell culture. These include plant-based like soy, wheat, rice (Lobo-Alfonso et al. 2008), and rapeseed (Farges-Haddani et al. 2006); animal-based such as Primatone R L (Schlaeger 1996), lactoalbumin hydrolysate (Mendonça et al. 2007), Casein hydrolysates (Phelan et al. 2009; Lahart et al. 2011) and microbial-based as yeast hydrolysate (Lobo-Alfonso et al. 2008; Mosser et al. 2013).

### Non-Mammalian Based Protein Hydrolysate in Animal Cell Culture (ACC)

Historically, mammalian derived hydrolysates were used extensively to replace serum. They are excellent source of proteins, peptides having bioactive properties and nourishment which makes it an ideal for serum substitution (Martinez-Alvarez et al. 2015). However, due to their taxonomic similarity to humans (Van Huis 2013), the risk of disease transmission is relatively high, which leads to their avoidance.

An animal tissue extract known as Primatone RL has been used as an inexpensive serum replacement in animal and murine cell cultures. This extract has a complex of many proteins, lipids, micronutrients and amino acids etc. Utilization of Primatone RL had been known to promote the growth of cells so that they achieve high cell densities, extended the life span of cells and elevated production of cell based products (Schlaeger 1996).

Lactalbumin is a milk-based protein (Buttriss 2003) that yields lactalbumin hydrolysate, which has been used in animal cell culture and cell line maintenance. A mitogen named LH-FI was isolated from it that was substituted for 10% FBS in Swiss 3T3 cells (Chou et al. 1979). Another major milk based protein hydrolysate, casein which is produced via pepsin and pancreatic digestion had also been used in animal based cell cultures (Nielsen et al. 2019). Jurkat T cells exhibited varying effects when supplemented with casein hydrolysate on its proliferation and viability (Phelan et al. 2009).

Despite established nutritional and growth inducing properties, mammalian-based hydrolysates have their limitations. Contamination introduced by mammalian-derived hydrolysates, which ultimately becomes part of the terminal product, is one of the main shortcomings that led the direction of animal-free media. Viral contamination is the primary concern posed by these hydrolysates restricting their use. (Merten 2002).

Certain novel sources may be able to overcome the disadvantages associated with conventional mammal-based hydrolysates. Among these lesser-known and used sources are protein hydrolysates derived from insects and marine invertebrates. Both of these non-mammalian sources are diverse, abundant, and high in protein (Oonincx et al. 2010; Gomez et al. 2019).

Insects belong to the largest class of animals, covering 95% of the biodiversity and were historically consumed at various stages of life (Anaya et al. 2013). There are many reasons insects make a good source of protein hydrolysate which assists in sustainability as well: 1) Insects have an excellent nutritional profile which consists high quantity of protein (50-71%), fats (13.4-33.4), and fibers (5.1-13.6) (Rumpold and Schlüter 2013) which validates them as a potential candidate for serum substitution . . . 2) Insects produce less amount of Green House Gases (GHGs) and ammonia compared to other animals, which leads to climate change and global warming (Oonincx et al. 2010) making their hydrolysate production more environmentally friendly in comparison to other animals. 3) Since humans and mammalian animals have a close taxonomic relationship, the risk of zoonotic diseases such as bovine spongiform encephalopathy (BSE) and avian influenza (H5N1) is high, rendering mammalian-based hydrolysate unsuitable for use, making insects an ideal protein hydrolysate source (Van Huis 2013). Black soldier fly (Hermetia illucens) and cricket (Gryllidae) are two widely accepted edible insects with high nutritional qualities (Bessa et al. 2020) that can produce high-quality protein hydrolysate that can be used in animal cell culture.

The marine world, which has a similar spectacular biodiversity, is a rich natural resource for a variety of biologically active compounds that have the potential to generate high-quality protein hydrolysates. Marine invertebrates are an excellent source of proteins and peptides, which can act as hormones, growth factors, and amino acids (Hamed et al. 2015). The oyster (Crassostrea virginica) is a vital marine bivalve, accounting for 33% of global production (Wijsman et al. 2019). It is a good source of protein, containing up to 80 percent protein on a dry weight basis (Gomez et al. 2019), making it an ideal candidate for the production of protein hydrolysates. Mussels (Mytilus edulis), another protein-rich bivalve, have been used to synthesize protein hydrolysate with bioactive properties such as antioxidant and cell-protective qualities (Oh et al. 2019). Another highly novel, protein-rich (Hirabayashi et al. 1998) and unexplored protein source is a marine invertebrate called the lugworm (Arenicola marina). Until now, the only successful application of lugworm protein hydrolysate has been documented in the treatment of damaged hair (Shin et al. 2015).

### Plant-Based Protein Hydrolysates in ACC

Hydrolysates derived from plants have been routinely used to reduce or eliminate serum from traditional basal media formulations, frequently in combination with a variety of other supplements (Babcock and Antosh 2012). Some successful examples of plant hydrolysates as serum substitutes are soy (Glycine max), wheat (Triticum), rice (Oryza sativa), and rapeseed (Brassica napus).

Soy hydrolysate is produced from soybeans that have high protein content of 40-50% with lipids and carbohydrates. Soybean has many storage proteins like albumins, glycinin and β-conglycinin. Production of soy hydrolysate requires enzymatic digestion by four enzymes that are pepsin, trypsin, papain and pancreatin (Zhang et al. 2010). Soy hydrolysates are known to increase cell proliferation, viability and protein production which makes it an effective replacement of serum. Apart from being a source of many macronutrients, soy hydrolysate has many micronutrients that can affect cell cultures like lactate, succinate and citrate (Gupta et al. 2015). Soy hydrolysate has been used in mammalian cell culture especially in Chinese Hamster Ovary (CHO) cells for production of recombinant protein production. In one study involving CHO-320 cells, soy hydrolysate exhibited marked elevation in cell growth and slight increase in interferon γ (γ-IFN) production. It was established that increase in γ-IFN was a result of rise in protein synthesis which was compromised via lowering of cell growth as well as escalation in secretion (Michiels et al. 2011).

Wheat hydrolysate is one of the most cost-effective proteins produced by the wheat industry that is rich source of many proteins and polypeptides. Production of wheat hydrolysate requires enzymatic action of various proteases namely alcalase, protamex, flavouzyme etc (Wang et al. 2007). One paper reported high cell growth and viability in mouse hybridoma (ME-750) cells and rise in monoclonal antibody (mAb) production when serum was substituted with wheat hydrolysate (Franěk 2004).

Rice hydrolysate is produced from rice bran in copious amounts by the agriculture industry. It has various macromolecules like carbohydrates, proteins, fibers and lipids (Tsigie et al. 2012). Rice is rich in many proteins like Albumins, globulins, prolamins and glutelins. Depending on the type of and part of the rice used, the biomolecular concentrations may vary. Many methods ranging from alkali extraction to enzymatic degradation have been applied in the production of rice hydrolysates (Hoogenkamp et al. 2017). One report which used serum-free media for CHO-320 cells described ameliorated production of γ-IFN as well as cell growth and proliferation (Bare et al. 2001).

Rapeseed is one of the most important crops used to produce oil. Rapeseed has decent proportion of amino acids as well as many other bioactive moieties like phenols, phylates and glucosinolates (Yoshie-Stark et al. 2008). Apart from these, rapeseed has cruciferin and napin that imparts it emulsifying and foaming properties which is required in animal cell culture. These moieties under pressure are also known to produce textures similar to meat (Kyriakopoulou et al. 2019). Rapeseed has only been recently suggested as an analogue to serum as compared to its other counterparts. One study that used CHO-C5 cell line for production of γ-IFN utilized serum free media with rapeseed peptide factions as a supplement. It showed enhancement of cell growth, which was not highly significant, but cell death was considerably reduced, however the reason for this was not known. Another factor they found was correlation between concentration of rapeseed peptide factions and cell growth, a higher concentration of these factions negatively impacted cell growth (Farges-Haddani et al. 2006).

Pea (Pisum sativum) is another highly promising and underutilized plant-based protein source. As a high-quality protein with a balanced amino acid ratio and all essential amino acids, pea protein could meet FAO/WHO recommendations. In addition, the lower allergenicity, non-GMO nature, higher nutritional value, and overall economic benefits have made its hydrolysate a potentially good option for serum substitution. (Ding et al. 2020).

### 2.4 Fungi and Algae Based Protein Hydrolysates in ACC

Numerous protein hydrolysates derived from fungi and algae have been used to replace serum in media, with yeast (Saccharomyces cerevisiae) being the most successful. These sources are naturally high in protein (typically greater than 30%), making them excellent candidates for protein hydrolysates. Additionally, their rapid growth rates and ability to grow on novel substrates make them commercially viable (Ritala et al. 2017).

Yeast hydrolysate is the most commonly used serum replacement in animal cell culture (Sung et al. 2004). Yeast hydrolysates are a concoction of many biomolecules like nucleic acids, carbohydrates, proteins, fats and other cellular components which makes it an excellent nutrient source (Kim and Lee 2009). Yeast extracts and yeast peptones had been used as media additive for IgG production for CHO-AMW cell line. These two were able to ameliorate cell growth and viability but exerted two different effects. Yeast extract was able to induce high cell growth which led to establishment of high cell density whereas yeast peptone enhanced IgG production. A future incentive for their further characterization was also given to fully understand these effects (Mosser et al. 2013).

Microalgae, specifically Chlorella vulgaris, is a single-celled organism that is highly nutritious due to its high protein content (>55% dry weight) and lipid-accumulating properties, earning it a place in the health supplement industry (Safi et al. 2014). In addition, it has recently been demonstrated to be an adequate serum substitute due to its cell proliferative properties (Song et al. 2012; Ng et al. 2020). However, most research has focused on extracts rather than protein hydrolysates.

Similarly, the button mushroom, or Agaricus bisporus, which is the most widely cultivated mushroom in the United States, has an excellent nutritional profile with an extremely high protein content comparable to animal sources. Apart from proteins, they are excellent source of fatty acids, phenolic componds, micro—and macronutrients (Atila et al. 2021). However, its potential for use as a serum substitute has not been investigated.

Even though all these novel sources have immensely beneficial chemical and biological composition, none of them have been intensively applied in animal cell culture systems. Despite of the fact that these sources show great promise as serum substitute, extensive research and evidence is required for their active utilization.

### Effect of Protein Hydrolysates on Growth and Biomass

Apart from their role of providing nourishment to the cells for growth, protein hydrolysates have shown many outcomes that suggest that their role is not limited to providing nutrients.
1) Due to absence of serum in serum free media, cells are exposed to additional stresses that leads to programmed cell death or apoptosis. This affects not only cell growth but production of cell-based products like monoclonal antibody. Hydrolysates are known to enhance cell viability by extending life span of cell by subduing apoptosis. Although the exact reason for this is not known, but it is proposed that peptides in the hydrolysate function as survival factor that are able to somehow suppress programmed cell death and induce cell growth as well as production of by-products (Franěk 2004).
2) Many growth factors are required for proliferation of cells like insulin and insulin like growth factors, epidermal growth factor etc. They are known to have “bioactive” properties which imparts them growth factor like properties. It is proposed that hydrolysates provide these growth actor like properties to the cell which in turn improves cell metabolic efficiency and resultantly improves cell growth (Burteau et al. 2003; Mosser et al. 2013).
3) As hydrolysates are source of many micronutrients like minerals, vitamins etc., these are well known to enhance oxidative metabolism. These micronutrients are required for functioning of many enzymes that take part in basal functioning of cells and thus increase cell viability and growth (Luo et al. 2012).
4) Some peptides in hydrolysates are known to increase the cell-based products by inhibiting cell growth. This shift is usually associated with the termination of exponential phase and onset of stationary phase that induces production of cell-based products. This process is usually related to the capacity of cell to consume lactate as an alternative source thus suppressing its toxic effect and maintaining overall productivity (Burky et al. 2007; Mosser et al. 2013).

In the Table 1 below, a brief account of protein hydrolysate utilization and the effects it exerted in animal cell culture is given below.

TABLE 1

| | | Resultant | |
|---|---|---|---|
| Protein | Type(s) | products | |
| hydrolysate(s) | of cell | of the animal | |
| used | cultivated | cell culture | References |
| Plant protein | Mouse hybridoma | Production of | (Franěk et |
| hydrolysate | cells (ME-750) | specific | al. 2000) |
| | | antibodies | |
| Yeastolate | Two recombinant | Biomass and | (Kim and |
| ultrafiltrate, soy | Chinese hamster | antibody | Lee 2009) |
| hydrolysate, | ovary cells | production | |
| wheat gluten | (rCHO) | | |
| hydrolysate | | | |
| and their blends | | | |
| Yeast | Channel | Biomass | (Radošević |
| hydrolysate; | catfish ovary | production | et al. |
| wheat gluten | (CCO) cell line | | 2016) |
| hydrolysate; | | | |
| Soy hydrolysate | | | |
| Soy protein | Chinese hamster | Biomass | (Gupta et |
| hydrolysate | ovary cells (CHO) | production | al. 2015) |
| Peptide fraction | Chinese hamster | Production of | (Farges- |
| of rapeseed | ovary cells (CHO) | specific | Haddani |
| hydolysate | | antibodies | et al. 2006) |
| Bacto-soytone | Human | Production of | (Lee et al. |
| and soy | keratinocytes | specific | 2008) |
| hyrdolysate | | antibodies | |
| | | Biomass | |
| | | production | |
| | | and maximum | |
| | | cell density | |
| Soybean | Human skin | Biomass | (Farizhandi |
| and rice | fibroblast | production and | et al. |
| grain protein | (HSF) | maximum cell | 2014) |
| hydrolysate | 1184 cells | density | |

As established from the table above, protein hydrolysates have been used successfully in many animal cell cultures for achieving high density biomass production as well as producing cell-based commodities In order to successfully commercialize and industrialize cultured meat, a media that boosts cell growth and proliferation without the use of serum is essential. This proposal brings us one step closer to that goal by incorporating a variety of protein hydrolysate sources.

### Production of Protein Hydrolysates

Protein hydrolysates can be synthesized in a variety of ways, including chemical processing, enzymatic hydrolysis, or microbial fermentation (Nasri 2017). Among the methods and according to the literature, in vitro hydrolysis of protein substrates using appropriate exogenous proteolytic enzymes is the widely used process to produce protein hydrolysates and peptides with desirable biological properties (Kristinsson & Rasco, 2000). Enzymatic proteolysis is better for producing protein hydrolysates than chemical treatments because the process conditions are milder (pH 6.0-8.0; temperature 40-60° C.), and the enzymatic hydrolysis is highly controlled. Furthermore, unlike chemical processes, the overall amino acid composition of enzymatic protein hydrolysates is nearly identical to that of the protein substrate, with slight modifications depending on the enzyme used. Enzymatic digestion does not use organic solvents or toxic chemicals, making it suitable for the food and pharmaceutical industries (Kim & Wijesekara, 2010). Enzymes from animal sources are more specific to their site of action compared to plant enzymes, which are more broadly specific in their action. Endopeptidases recognize specific amino acids in the middle of the peptide, whereas exopeptidases recognize one or two terminal amino acids. In the recent studies endopeptidases especially Alcalase is the most common and efficacious enzyme employed for the synthesis of protein hydrolysates (Marson et al. 2020).

### Materials and Methods

Production and Characterization of Protein Hydrolysates from Various Marine, Insect, Plant, and Single-Cell Sources

The uniqueness, nutrient composition, protein content, and availability of various raw materials were considered when selecting them for protein hydrolysate production. In addition to raw materials-enzymes, hydrolysis time, and physicochemical conditions such as pH and temperature contribute to the production and fabrication of protein hydrolysates (Leni et al. 2020). Thus, for maximum efficacy, protein hydrolysates were methodically produced and characterized using the following eight raw materials: Insect based substrate: Black soldier Fly (Hermetia illucens) and Cricket (Gryllidae); Marine based substrate: Oyster (Crassostrea virginica), Mussel (Mytilus edulis), and Lugworm (Arenicola marina).; Plant based substrate: Pea protein (Pisum sativum); Algae and Fungi-based substrates: Microalgae (Chlorella vulgaris) and Button mushroom (Agaricus bisporus).

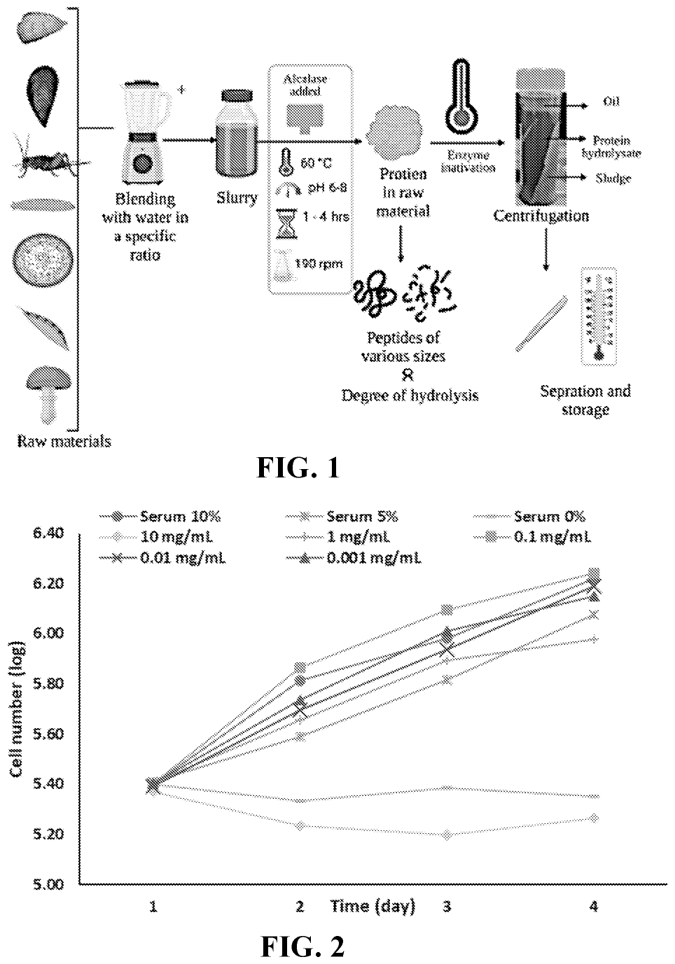

The overall process of hydrolyzing protein sources is summarized in FIG. 1. To initiate the protein hydrolysis process, a slurry of raw materials containing a specified amount of water would be prepared by grinding. This step ensures the uniform distribution of the enzyme throughout the system via water. Then, optimal conditions for the proteolytic enzyme—Alcalase was generated are a temperature (60° C.) and pH (6-8) for the slurry produced. Enzymes were used that hydrolyze proteins hydrolyze effectively at the optimal temperature and pH, typically cleaving specific peptide bonds, resulting in the digestion of protein and production of various amino acids and peptides. Following that, the reaction vessel was stirred continuously to ensure that the enzyme is distributed evenly and remains active for the reaction duration. Without being bound by theory, enzymatic hydrolysis improves protein functionality by exposing the protein structure, lowering the average molecular weight, and increasing ionic strength, molecular charges, and protein-protein interactions. Once the reaction time has ended, the elevated temperature inactivates heat liable enzymes by heating the slurry to a higher temperature (90° C.). Finally, centrifugation was used separate the hydrolysate, lyophilized, and stored at −20° C. until further experimentation. (Kristinsson and Rasco 2000; Zhu et al. 2020).

Characterization of the obtained protein hydrolysate is important for ensuring that the process was successful and optimizing the hydrolysate's required functional properties (Saadi et al. 2015). For this study, cell proliferation and viability were the essential characteristics necessary for the active property of protein hydrolysate. The protein hydrolysates were characterized by following criteria: Yield—The term refers to the amount of product formed per unit of substrate supplied. The following formulae (Eq. 1) was used to determine the yield of the protein hydrolysate (Firmansyah and Abduh 2019):

$$Yield ( \% ) = \frac{W eight of lyophilized protein hydrolysate ( g )}{W eight of raw material used ( g )} \times 100 \qquad ( Eq . 1 )$$

Productivity—This is expressed in terms of the amount of protein hydrolysate produced per milliliter of batch reaction (Firmansyah and Abduh 2019). The following formulae (Eq. 2) was used to determine the productivity of protein hydrolysate:

$$Productivity ( \frac{mg}{ml} ) = \frac{weight of lyophilize d protein hydrolysate ( mg )}{r e a ction volume ( mL )} \qquad ( Eq . 2 )$$

Degree of hydrolysis (DH)-DH of a protein hydrolysate is defined as the proportion of cleaved peptide bonds (Rutherfurd 2010). The degree of hydrolysis was determined in triplicate using formol titration as the ratio of amino groups released per total nitrogen sample indicated by lowering of pH as a proton is released (Taylor 1957).

Amino acid composition—The structure of protein hydrolysates, as well as their functional and bioactive properties, are highly dependent on their amino acid composition and sequence, making their characterization important (Sánchez and Vázquez 2017). Amino acid analysis can be outsourced to CVAS laboratory in which the composition can be determined according to the methodology provided by Association of Official Agricultural Chemists (AOAC) (Parliament et al. 2009).

### Determine the Techno-Functional Properties of the Protein Hydrolysates.

Techno-functional properties are important for protein hydrolysates in food technology because they define their hydrophilic or phobic nature and their ability to form emulsions and foams (Téllez-Morales et al. 2020). Essentially, their interaction with other components in the system is determined by their technofunctional properties, which makes them critical for testing. The following properties were assessed for the protein hydrolysates:

Oil holding capacity (OIC)—This will be determined according to the method followed by Shahidi et al. 1995. The protein hydrolysate sample will be mixed with pure canola oil and incubated at room temperature for 30 minutes. Following that, the free oil will be extracted, and the OIC would be determined by weighing.

Emulsifying capacity (EC)—This will be determined according to Yasumatsu et al. 1972 method. The formulae used to calculate ES is as follows (Eq. 3):

$$Emulsyfying capacity ( EC ( \% ) ) = \frac{Total volume - A q ueous volume}{total volume} \times 100 \qquad ( Eq . 3 )$$

Foaming capacity (FC)-Aeration method was followed for calculating FC of the protein hydrolysate samples (Pacheco-Aguilar et al. 2008). The formulae used to calculate FC was as follows (Eq. 4):

$$Foaming capacity ( FC ( \% ) ) = \frac{Aerated volume - N ormal volume}{N ormal volume} \times 100 \qquad ( Eq . 4 )$$

### Evaluating the Impact of Different Concentrations of Protein Hydrolysates on Cell Performance Under Serum-Free and Reduced-Serum Conditions.

Many research articles indicate that protein hydrolysates may be used in place of serum because they provide energy and exhibit growth factor-like properties due to the 3-D conformation of peptides. (Sung et al. 2004; Ganglberger et al. 2007; Lobo-Alfonso et al. 2008; Andreassen et al. 2020; Logarušić et al. 2021). Essentially, both free amino acids and peptides of variable length are required to promote cell growth and proliferation (Lobo-Alfonso et al. 2008). Many recent studies have elucidated various protein hydrolysate concentrations that augment the cell growth and proliferation in concentrations ranging from 10 mg/ml-0.001 mg/mL (Cancre et al. 1999; Phelan et al. 2009; Kim et al. 2011; Behera et al. 2013; Radošević et al. 2016; Andreassen et al. 2020; Chotphruethipong et al. 2021; Logarušić et al. 2021). The use of protein hydrolysate has many practical constraints, including the wide range of concentrations used, the presence of unknown bioactive components, and a lack of specific knowledge regarding the effect of protein hydrolysate on fish cells. As a result, it becomes important to determine the optimal hydrolysate dosage for a given medium formulation in order to achieve the desired effects, such as increased cell growth and viability, experimentally (Logarušić et al. 2021).

Five different hydrolysate concentrations were tested-10 mg/mL, 1 mg/mL, 0.1 mg/mL, 0.01 mg/mL, 0.001 mg/mL with 10%, 5% and 0% serum conditions. The model zebrafish embryonic stem cell line (ZEM2S) from ATCC was used for these studies. Ideally, a certain concentration of protein hydrolysates should be able to completely substitute serum in serum free conditions, or at least restore a part of the lost serum function in serum reduced conditions (Lobo-Alfonso et al. 2008; Andreassen et al. 2020). All tests were conducted with positive (10% serum-containing), reduced (5% serum containing) and negative (basal media) controls in tissue culture (TC)-treated plates with four biological replicates with three technical replicates each.

The success of the protein hydrolysates was determined by cell growth, confluence, and viability in comparison to the controls. The cell growth will be monitored daily by Olympus inverted microscope CKX53 with CKX-CCSW software for counting cells and confluency. Viability of the cells will be determined by a cell viability colorimetric agent-Presto Blue.

### REFERENCES RELATED TO EXAMPLE 1

Anaya J-M, Shoenfeld Y, Rojas-Villarraga A, et al (2013) Autoimmunity: from bench to bedside. El Rosario University Press.
Andreassen R C, Pedersen M E, Kristoffersen K A, Rønning S B (2020) Screening of by-products from the food industry as growth promoting agents in serum-free media for skeletal muscle cell culture. Food Funct 11:2477-2488.
Atila F, Owaid M N, Shariati M A (2021) The nutritional and medical benefits of Agaricus bisporus: a review. J Microbiol Biotechnol Food Sci 2021:281-286.
Babcock J F, Antosh A (2012) Partial replacement of chemically defined CHO media with plant-derived protein hydrolysates. In: Proceedings of the 21st Annual Meeting of the European Society for Animal Cell Technology (ESACT), Dublin, Ireland, Jun. 7-10, 2009. Springer, pp 295-298.
Bare G, Charlier H, De Nijs L, et al (2001) Effects of a rice protein hydrolysate on growth of CHO cells and production of human interferon-γ in a serum-free medium. In: Animal Cell Technology: from target to market. Springer, pp 217-219.
Behera P, Kumar R, Sandeep IVR, et al (2013) Casein hydrolysates enhance osteoblast proliferation and differentiation in mouse bone marrow culture. Food Biosci 2:24-30.
Bessa L W, Pieterse E, Marais J, Hoffman LC (2020) Why for feed and not for human consumption? The black soldier fly larvae. Compr Rev Food Sci Food Saf 19:2747-2763.
Bhat S, Viswanathan P, Chandanala S, et al (2021) Expansion and characterization of bone marrow derived human mesenchymal stromal cells in serum-free conditions. Sci Rep 11:3403. https://doi.org/10.1038/s41598-021-83088-1
Brunner D, Frank J, Appl H, et al (2010) Serum-free cell culture: the serum-free media interactive online database. ALTEX 27:53-62. https://doi.org/10.14573/alt
Burky JE, Wesson MC, Young A, et al (2007) Protein-free fed-batch culture of non-GS NSO cell ex.2010.1.53 lines for production of recombinant antibodies. Biotechnol Bioeng 96:281-293
Burteau C C, Verhoeye F R, Molsl J F, et al (2003) Fortification of a protein-free cell culture medium with plant peptones improves cultivation and productivity of an interferon-γ-producing CHO cell line. Vitr Cell Dev Biol 39:291-296
Buttriss J (2003) MILK| dietary importance
Cancre I, Ravallec R, Van Wormhoudt A, et al (1999) Secretagogues and growth factors in fish and crustacean protein hydrolysates. Mar Biotechnol 1:489-494
Chotphruethipong L, Binlateh T, Hutamekalin P, et al (2021) Impact of Hydrolyzed Collagen from Defatted Sea Bass Skin on Proliferation and Differentiation of Preosteoblast MC3T3-El Cells. Foods 10:1476
Chou IN, Prezyna C, Black PH (1979) Isolation from lactalbumin hydrolysate of a high molecular weight mitogenic factor. J Biol Chem 254:10588-10591
Ding J, Liang R, Yang Y, et al (2020) Optimization of pea protein hydrolysate preparation and purification of antioxidant peptides based on an in silico analytical approach. LWT 123:109126. https://doi.org/https://doi.org/10.1016/j.lwt.2020.109126.
Farges-Haddani B, Tessier B, Chenu S, et al (2006) Peptide fractions of rapeseed hydrolysates as an alternative to animal proteins in CHO cell culture media. Process Biochem 41:2297-2304
Farizhandi A A K, Rahman S A S A, Majid FAA, et al (2014) PLANT PROTEIN HYDROLYSATES FROM SOY BEAN AND RICE GRAIN AS A SUPPLEMENT FOR MEDIUM IN HUMAN SKIN FIBROBLAST 1184 CELL CULTURE. Adv Environ Biol 838-844
Firmansyah M, Abduh M Y (2019) Production of protein hydrolysate containing antioxidant activity from Hermetia illucens. Heliyon 5: e02005
Franěk F (2004) Gluten of spelt wheat (Triticum aestivum subspecies spelta) as a source of peptides promoting viability and product yield of mouse hybridoma cell cultures. J Agric Food Chem 52:4097-4100
Franěk F, Hohenwarter O, Katinger H (2000) Plant protein hydrolysates: preparation of defined peptide fractions promoting growth and production in animal cells cultures. Biotechnol Prog 16:688-692
Freshney R I (2016) Culture of animal cells: a manual of basic technique and specialized applications, Seventh ed. Wiley Blackwell, Hoboken, New Jersey
Freshney R I (2015) Culture of animal cells: a manual of basic technique and specialized applications. John Wiley & Sons
Galbraith S C, Bhatia H, Liu H, Yoon S (2018) Media formulation optimization: current and future opportunities. Curr Opin Chem Eng 22:42-47
Ganglberger P, Obermüller B, Kainer M, et al (2007) Optimization of Culture Medium with the Use of Protein Hydrolysates. In: Smith R (ed) Cell Technology for Cell Products. Springer Netherlands, Dordrecht, pp 553-557
Gomez H L R, Peralta J P, Tejano L A, Chang Y-W (2019) In silico and in vitro assessment of portuguese oyster (Crassostrea angulata) proteins as precursor of bioactive peptides. Int J Mol Sci 20:5191
Gupta A J, Wierenga P A, Gruppen H, Boots J (2015) Influence of protein and carbohydrate contents of soy protein hydrolysates on cell density and IgG production in animal cell cultures. Biotechnol Prog 31:1396-1405
Hamed I, Özogul F, Özogul Y, Regenstein J M (2015) Marine bioactive compounds and their health benefits: a review. Compr Rev food Sci food Saf 14:446-465
Hirabayashi J, Dutta SK, Kasai K (1998) Novel galactose-binding proteins in Annelida: Characterization of 29-kDa tandem repeat-type lectins from the earthworm Lumbricus terrestris. J Biol Chem 273:14450-14460
Hoogenkamp H, Kumagai H, Wanasundara J P D (2017) Rice protein and rice protein products. In: Sustainable Protein Sources. Elsevier, pp 47-65
Karnieli O, Friedner O M, Allickson J G, et al (2017) A consensus introduction to serum replacements and serum-free media for cellular therapies. Cytotherapy 19:155-169
Kim J Y, Kim Y-G, Han Y K, et al (2011) Proteomic understanding of intracellular responses of recombinant Chinese hamster ovary cells cultivated in serum-free medium supplemented with hydrolysates. Appl Microbiol Biotechnol 89:1917-1928
Kim S H, Lee G M (2009) Development of serum-free medium supplemented with hydrolysates for the production of therapeutic antibodies in CHO cell cultures using design of experiments. Appl Microbiol Biotechnol 83:639-648
Kristinsson HG, Rasco BA (2000) Fish protein hydrolysates: production, biochemical, and functional properties. Crit Rev Food Sci Nutr 40:43-81
Kuo H-H, Gao X, DeKeyser J-M, et al (2020) Negligible-Cost and Weekend-Free Chemically Defined Human iPSC Culture. Stem cell reports 14:256-270. https://doi.org/10.1016/j.stemcr.2019.12.007
Kyriakopoulou K, Dekkers B, van der Goot A J (2019) Plant-based meat analogues. In: Sustainable Meat Production and Processing. Elsevier, pp 103-126
Lee Y K, Kim S Y, Kim K H, et al (2008) Use of soybean protein hydrolysates for promoting proliferation of human keratinocytes in serum-free medium. Biotechnol Lett 30:1931-1936
Leni G, Soetemans L, Caligiani A, et al (2020) Degree of Hydrolysis Affects the Techno-Functional Properties of Lesser Mealworm Protein Hydrolysates. Foods 9:381
Ling W L W, Bai Y, Cheng C, et al (2015) Development and manufacturability assessment of chemically-defined medium for the production of protein therapeutics in CHO cells. Biotechnol Prog 31:1163-1171. https://doi.org/10.1002/btpr.2108
Lobo-Alfonso J, Price P, Jayme D (2008) Benefits and limitations of protein hydrolysates as components of serum-free media for animal cell culture applications. In: Protein Hydrolysates in Biotechnology. Springer, pp 55-78
Logarušić M, Gaurina Srček V, Berljavac S, et al (2021) Protein Hydrolysates from Flaxseed Oil Cake as a Media Supplement in CHO Cell Culture. Resour. 10
Luo J, Vijayasankaran N, Autsen J, et al (2012) Comparative metabolite analysis to understand lactate metabolism shift in Chinese hamster ovary cell culture process. Biotechnol Bioeng 109:146-156
Marson G V, de Castro R J S, da Costa Machado M T, et al (2020) Proteolytic enzymes positively modulated the physicochemical and antioxidant properties of spent yeast protein hydrolysates. Process Biochem 91:34-45
Martinez-Alvarez O, Chamorro S, Brenes A (2015) Protein hydrolysates from animal processing by-products as a source of bioactive molecules with interest in animal feeding: A review. Food Res Int 73:204-212
Merten O W (2002) Development of serum-free media for cell growth and production of viruses/viral vaccines--safety issues of animal products used in serum-free media. Dev Biol (Basel) 111:233-257
Michiels J-F, Sart S, Schneider Y-J, Agathos S N (2011) Effects of a soy peptone on γ-IFN production steps in CHO-320 cells. Process Biochem 46:1759-1766
Mosser M, Chevalot I, Olmos E, et al (2013) Combination of yeast hydrolysates to improve CHO cell growth and IgG production. Cytotechnology 65:629-641
Nasri M (2017) Protein hydrolysates and biopeptides: Production, biological activities, and applications in foods and health benefits. A review. In: Advances in food and nutrition research. Elsevier, pp 109-159
Ng J Y, Chua M L, Zhang C, et al (2020) Chlorella vulgaris extract as a serum replacement that enhances mammalian cell growth and protein expression. Front Bioeng Biotechnol 8:1068
Nielsen S D, Purup S, Larsen L B (2019) Effect of Casein Hydrolysates on Intestinal Cell Migration and Their Peptide Profiles by LC-ESI/MS/MS. Foods 8:91
Oh Y, Ahn C-B, Nam K-H, et al (2019) Amino acid composition, antioxidant, and cytoprotective effect of blue mussel (Mytilus edulis) hydrolysate through the inhibition of caspase-3 activation in oxidative stress-mediated endothelial cell injury. Mar Drugs 17:135
Oonincx D G A B, Van Itterbeeck J, Heetkamp M J W, et al (2010) An exploration on greenhouse gas and ammonia production by insect species suitable for animal or human consumption. PLOS One 5: e14445
Pacheco-Aguilar R, Mazorra-Manzano M A, Ramírez-Suárez J C (2008) Functional properties of fish protein hydrolysates from Pacific whiting (Merluccius productus) muscle produced by a commercial Food protease. Chem 109:782-789. https://doi.org/10.1016/j.foodchem.2008 Parliament THEE, Council THE, The OF, Union E (2009) 1.9.2009.1-28.01.047
Phelan M, Aherne-Bruce S A, O'Sullivan D, et al (2009) Potential bioactive effects of casein hydrolysates on human cultured cells. Int Dairy J 19:279-285
Radošević K, Dukić B, Andlar M, et al (2016) Adaptation and cultivation of permanent fish cell line CCO in serum-free medium and influence of protein hydrolysates on growth performance. Cytotechnology 68:115-121
Ritala A, Häkkinen S T, Toivari M, Wiebe M G (2017) Single Cell Protein-State-of-the-Art, Industrial Landscape and Patents 2001-2016. Front. Microbiol. 8:2009
Rumpold B A, Schlüter O K (2013) Nutritional composition and safety aspects of edible insects. Mol Nutr Food Res 57:802-823
Rutherfurd S M (2010) Methodology for DeterminingDegree of Hydrolysis of Proteins in Hydrolysates: A Review. J AOAC Int. 93:1515-1522. https://doi.org/10.1093/jaoac/93.5.1515
Saadi S, Saari N, Anwar F, et al (2015) Recent advances in food biopeptides: production, biological functionalities and therapeutic applications. Biotechnol Adv 33:80-116. https://doi.org/10.1016/j.biotechadv.2014.12.003
Safi C, Zebib B, Merah O, et al (2014) Morphology, composition, production, processing and applications of Chlorella vulgaris: A review. Renew Sustain Energy Rev 35:265-278. https://doi.org/https://doi.org/10.1016/j.rser.2014.04.007
Sánchez A, Vázquez A (2017) Bioactive peptides: A review. Food Qual Saf 1:29-46. https://doi.org/10.1093/fqsafe/fyx006
Schlaeger E-J (1996) The protein hydrolysate, Primatone RL, is a cost-effective multiple growth promoter of mammalian cell culture in serum-containing and serum-free media and displays anti-apoptosis properties. J Immunol Methods 194:191-199
Shahidi F, Han X-Q, Synowiecki J (1995) Production and characteristics of protein hydrolysates from capelin (Mallotus villosus). Food Chem 53:285-293
Shin J E, Kang S M, Koh J, Kwon Y J (2015) Effect of lugworm protein hydrolysates containing proteins of differing molecular weight distributions on permanent wave treatment in damaged hair. Mater Res Innov 19: S5-1009
Sinacore M S, Drapeau D, Adamson SR (2000) Adaptation of mammalian cells to growth in serum-free media. Mol Biotechnol 15:249-257
Song S-H, Kim I-H, Nam T-J (2012) Effect of a hot water extract of Chlorella vulgaris on proliferation of IEC-6 cells. Int J Mol Med 29:741-746
Sung Y H, Lim S W, Chung J Y, Lee G-M (2004) Yeast hydrolysate as a low-cost additive to serum-free medium for the production of human thrombopoietin in suspension cultures of Chinese hamster ovary cells. Appl Microbiol Biotechnol 63:527-536
Taylor W G, Dworkin R A, Pumper R W, Evans V J (1972) Biological efficacy of several commercially available peptones for mammalian cells in culture. Exp Cell Res 74:275-279
Taylor W G, Evans V J, Pumper R W (1974) Studies on a serum substitute for mammalian cells in culture. In Vitro 9:278-285
Taylor W H (1957) Formol titration: an evaluation of its various modifications. Analyst 82:488-498
Téllez-Morales J A, Hernández-Santo B, Rodríguez-Miranda J (2020) Effect of ultrasound on the techno-functional properties of food components/ingredients: A review. Ultrason Sonochem 61:104787. https://doi.org/https://doi.org/10.1016/j.ultsonch.2019.104787
Tsigie Y A, Wang C-Y, Kasim N S, et al (2012) Oil production from Yarrowia lipolytica Polg using rice bran hydrolysate. Biomed Res Int 2012.
van der Valk J, Brunner D, De Smet K, et al (2010) Optimization of chemically defined cell culture media-Replacing fetal bovine serum in mammalian in vitro methods. Toxicol Vitr 24:1053-1063. https://doi.org/10.1016/j.tiv.2010.03.016
Van Huis A (2013) Potential of insects as food and feed in assuring food security. Annu Rev Entomol 58:563-583
Wang J, Zhao M, Zhao Q, et al (2007) Characterization of hydrolysates derived from enzymatic hydrolysis of wheat gluten. J Food Sci 72: C103-C107
Wijsman J W M, Troost K, Fang J, Roncarati A (2019) Global production of marine bivalves. Trends and challenges. In: Goods and services of marine bivalves. Springer, Cham, pp 7-26
Yao T, Asayama Y (2017) Animal-cell culture media: history, characteristics, and current issues. Reprod Med Biol 16:99-117
Yasumatsu K, Sawada K, Moritaka S, et al (1972) Whipping and emulsifying properties of soybean products. Agric Biol Chem 36:719-727
Yoshie-Stark Y, Wada Y, Wäsche A (2008) Chemical composition, functional properties, and bioactivities of rapeseed protein isolates. Food Chem 107:32-39
Zhang L, Li J, Zhou K (2010) Chelating and radical scavenging activities of soy protein hydrolysates prepared from microbial proteases and their effect on meat lipid peroxidation. Bioresour Technol 101:2084-2089
Zhu D, Huang X, Tu F, et al (2020) Preparation, antioxidant activity evaluation, and identification of antioxidant peptide from black soldier fly (Hermetia illucens L.) larvae. J Food Biochem 44: e13186. https://doi.org/10.1111/jfbc.13186

### Example 2-Developing Serum Free Media Using Natural Based Peptides

A yeast hydrolysate and BSF hydrolysate were generated. The protein hydrolysates were added to the media at different concentrations including 0.001, 0.01, 0.1, 1, and 10 mg/mL. Positive controls were: media with 5 and 10% serum. Negative control was: media with 0% serum. The protein hydrolysates were added to 0, 5, and 10% serum-based media.

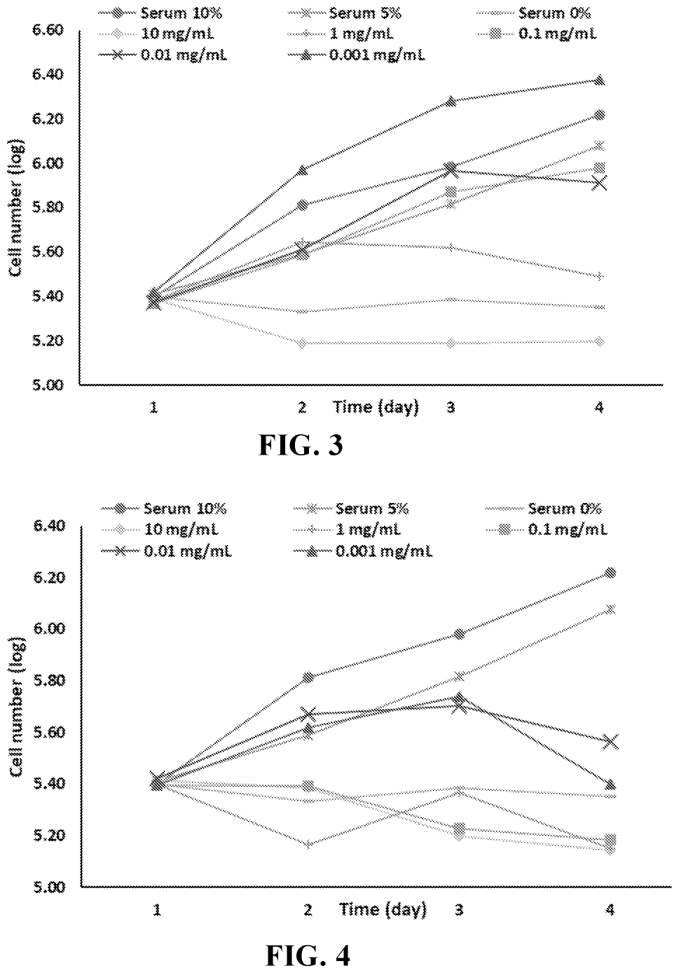

FIG. 2 shows results of cell culture with yeast hydrolysate+media with 10% serum. Adding 0.1 and 0.01 mg/ml yeast hydrolysate (YH) to the media containing 10% serum enhanced cell growth slightly compared to the media with 10% serum. YH at 10 mg/ml was toxic for the cells, and the growth was reduced compared to the 0% serum.

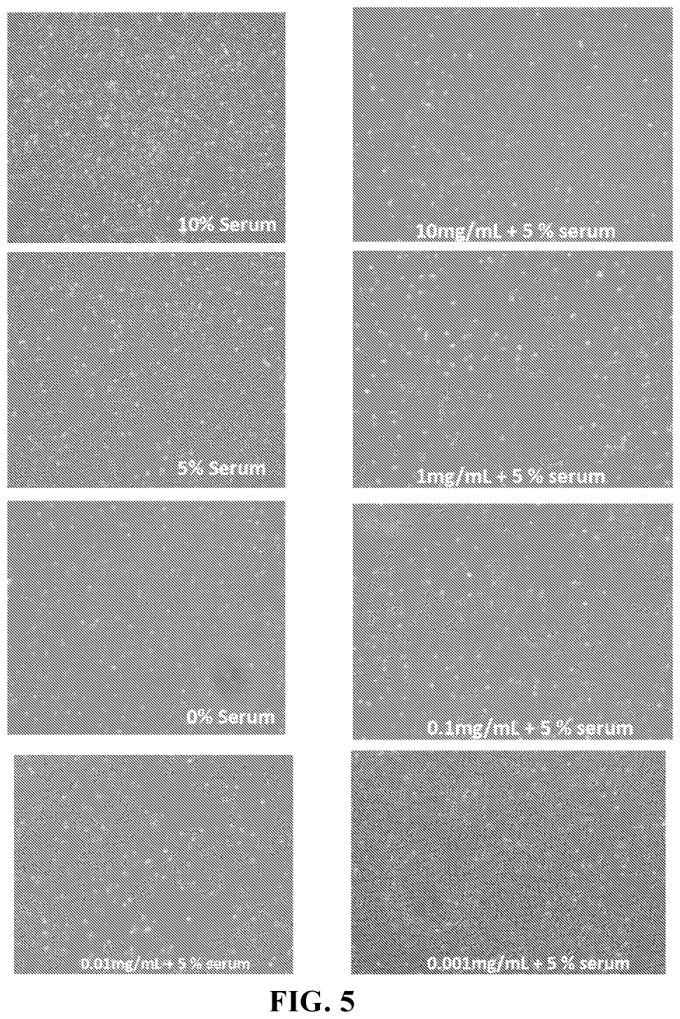

FIG. 3 shows results of cell culture with yeast hydrolysate+media with 5% serum. Adding 0.001 mg/ml YH to the media containing 5% serum enhanced cell growth slightly compared to the media with 10% and 5% serum. YH at 10 mg/ml was toxic for the cells, and the growth was reduced compared to the 0% serum.

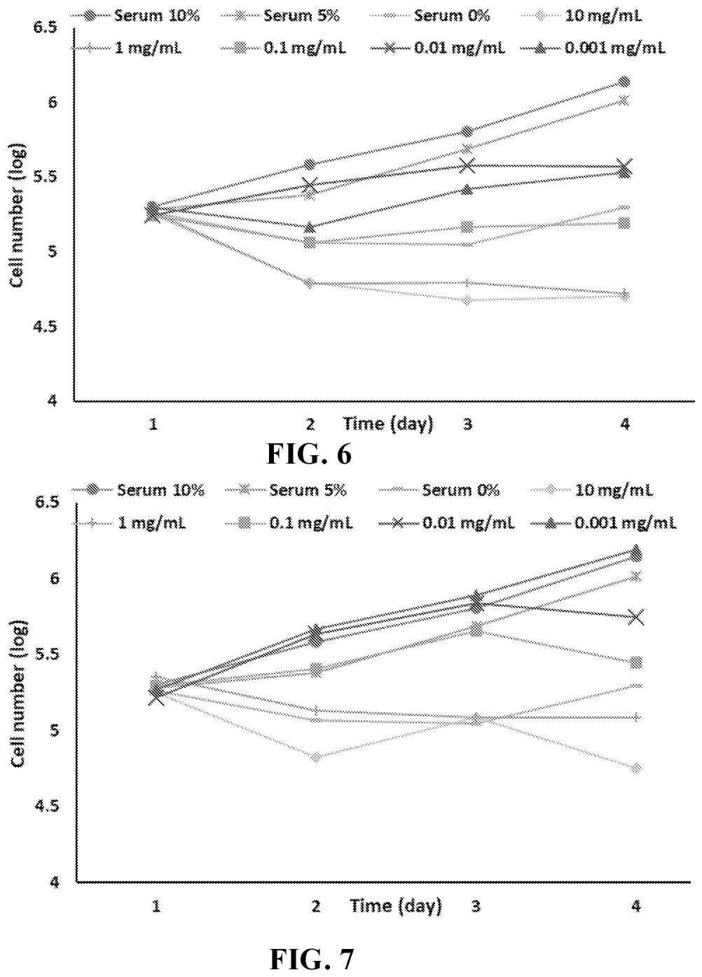

FIG. 4 shows results of cell culture with yeast hydrolysate+media with 0% serum. Adding 0.01 mg/ml and 0.001 mg/ml YH to the media containing 0% serum provided enough nutrients for the cell growth up to 3 days, and after that, slight cell growth was reduced. YH at 10 mg/ml was toxic for the cells, and the growth was reduced compared to the 0% serum.

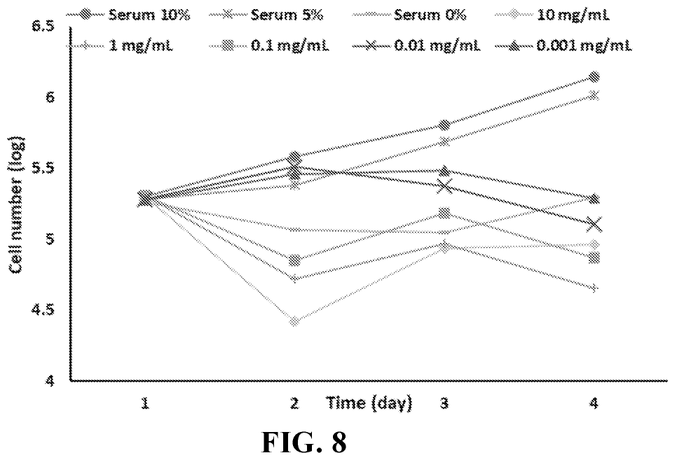

FIG. 5 shows phase contrast microscopic images of cells cultured in various yeast hydrolysate amounts with and with serum.

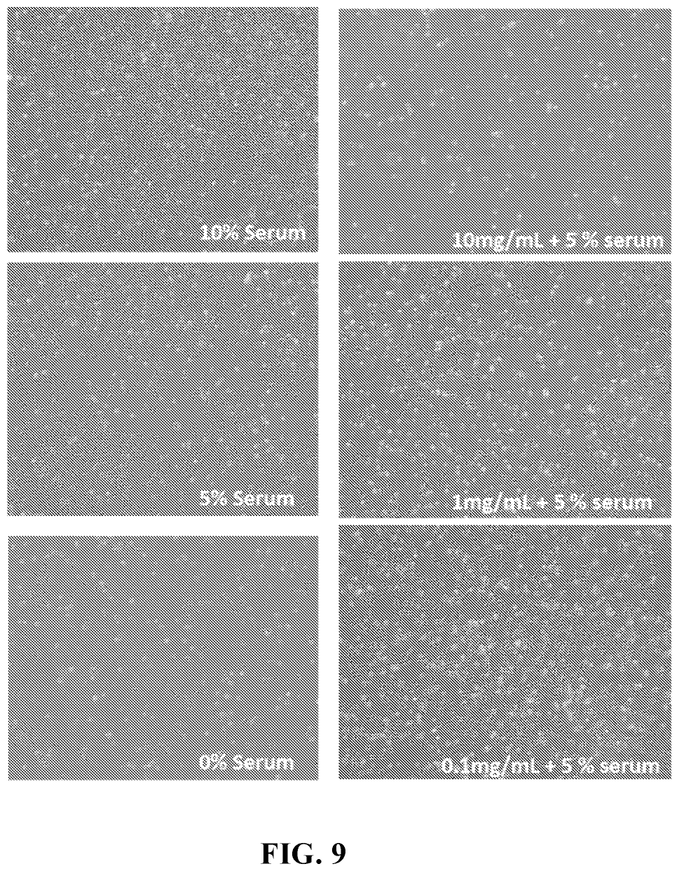

FIG. 6 shows results of cell culture with Black Soldier Fly (BSF), a representative insect, hydrolysate (BSFH)+media with 10% serum. Adding 0.01 and 0.001 mg/ml BSFH to the media containing 10% serum slightly reduced the cell growth. BSFH at 10, 1 and 0.1 mg/ml was toxic for the cells, and the growth was reduced compared to the 0% serum.

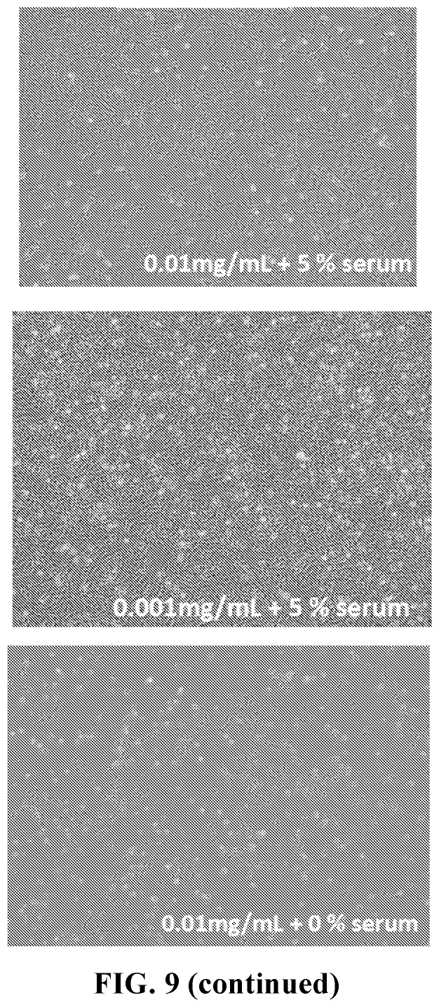

FIG. 7 shows results of cell culture with BSF hydrolysate (BSFH)+media with 5% serum. Adding 0.001 mg/ml BSFH to the media containing 5% serum enhanced the cell growth compared to the 5% serum, indicating that we can reduce the serum to 5% and get similar growth as we observed in media with 10% serum. BSFH at 10, and 1 mg/ml was toxic for the cells, and the growth was reduced compared to the 0% serum.

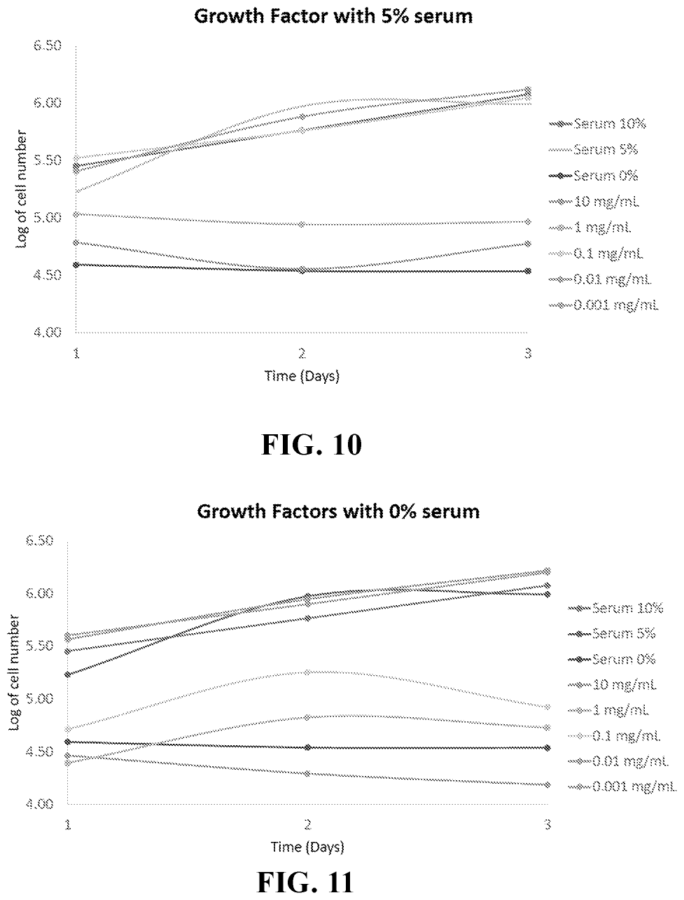

FIG. 8 shows results of cell culture with BSF hydrolysate+media with 0% serum. Adding 0.001 and 0.01 mg/ml BSFH to the media containing 0% provide enough peptides for cell growth until day 3 without changing the media. BSFH at 10, and 1 mg/ml was toxic for the cells, and the growth was reduced compared to the 0% serum.

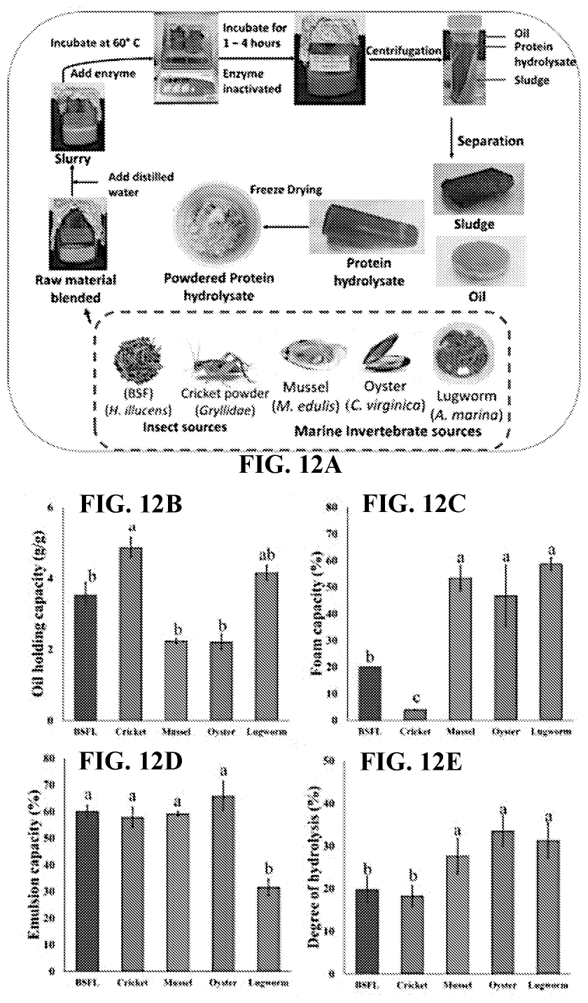

FIG. 9 shows phase contrast microscopic images of cells cultured in various BSF hydrolysate amounts with and with serum.

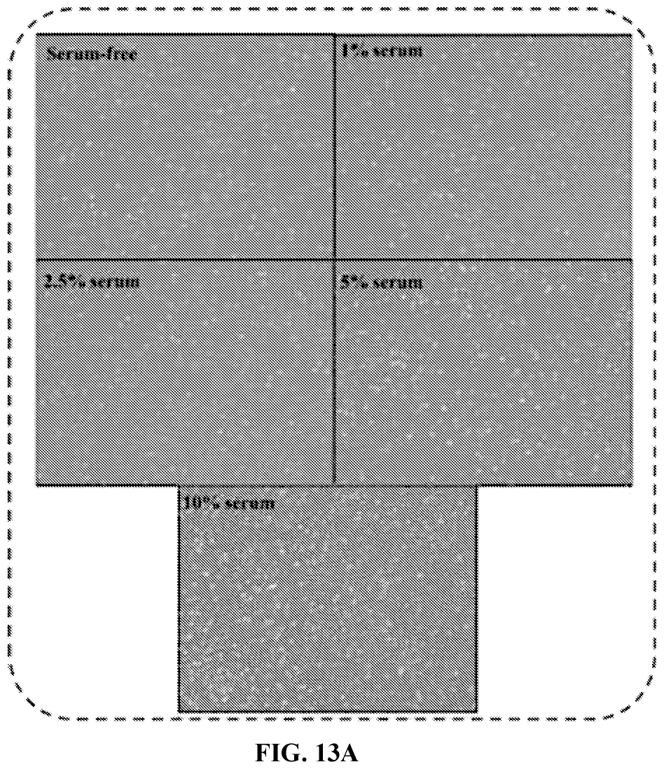

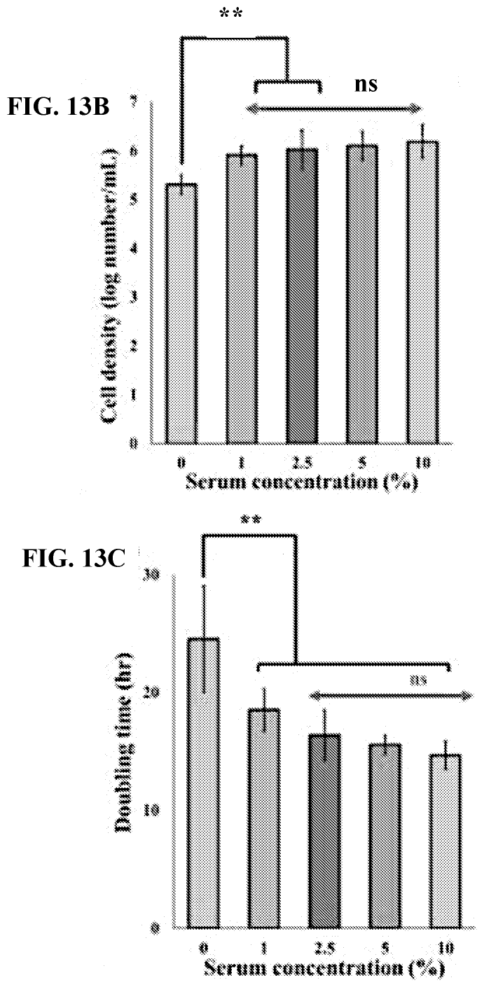

The effect of growth factors on cell culture growth was evaluated. FIG. 10 shows the effect of growth factors in media with 5% serum. FIG. 11 shows the effect of growth factors in media with 0% serum.

### Example 3-Evaluating the Potential of Marine Invertebrate and Insect Protein Hydrolysates to Reduce Fetal Bovine Serum in Cell Culture Media for Cultivated Fish Production

The use of fetal bovine serum (FBS) and the price of the cell culture media are the key constraints for developing serum-free cost-effective media. This study aims to replace or reduce the typical 10% serum application in fish cell culture media by applying protein hydrolysates from insects and marine invertebrate species for the growth of Zebrafish embryonic stem cells (ESC) as the model organism. Protein hydrolysates were produced from Black soldier fly (BSF), cricket, oyster, mussel, and lugworm with high protein content, suitable functional properties, adequate amino acids composition, and the degree of hydrolysis from 18.24 to 33.52%. Protein hydrolysates at low concentrations from 0.001 to 0.1 mg/mL in combination with 1 and 2.5% serum significantly increased cell growth compared to the control groups (5 and 10% serum) (P<0.05). All protein hydrolysates with concentrations of 1 and 10 mg/mL were found to be toxic to cells and significantly reduced cell growth and performance (P<0.05). However, except for cricket, all hydrolysates were able to restore or significantly increase cell growth and viability with 50% less serum at a concentration of 0.001, 0.01, and 0.1 mg/mL. Although cell growth was enhanced at lower concentrations of protein hydrolysates, cell morphology was altered due to the lack of serum. Lactate dehydrogenase (LDH) activity results indicated that BSF and lugworm hydrolysates did not alter the cell membrane. In addition, light and fluorescence imaging revealed that cell morphological features were comparable to the 10% serum control group. Overall, lugworm and BSF hydrolysates reduced serum by up to 90% while preserving excellent cell health.

### INTRODUCTION

The world population is projected to reach 10 billion by 2050, requiring a 70% increase in meat production to meet global demand (FAO, 2020). Animal-based products, especially meats, make up a substantial portion of global protein consumption (Whitnall and Pitts, 2019). Among animal-based meat, aquatic food products constitute 20% of the total animal protein consumed currently, and this consumption has increased rapidly from previous years (FAO, 2020). Conventionally, aquatic food products are obtained by fisheries or from different aquaculture systems, which are facing many challenges, including but not limited to overfishing, fraud, by—catch, antibiotic-resistant bacteria, emerging diseases, microplastics, and pollution (Smith et al., 2010; Cole et al., 2009). Cultivated meat appears to be a sustainable technique compared to conventional meat, with many environmental, economic, and health benefits over other conventional alternatives (van der Weele et al., 2019). Cultivated meat involves culturing cells or tissues in a specifically formulated media that promote cell proliferation, metabolism, and differentiation (Bhatia et al., 2019). Cell culture media accounts for 85-95% of the total cost of the cultivated meat process (Stout et al., 2021), and Fetal Bovine Serum (FBS) is one of the most expensive components of cell culture media. FBS has critical roles by supporting cell proliferation and metabolism. Due to high cost, risk of disease, high demand-less supply, high variability, inability to grow specific cells, and ethical source issues (Stout et al., 2021), developing serum-free media for the cultivated meat industry could reduce the cost of the final products and address sustainability concerns. Despite successful efforts to develop serum-free media for medical applications, the high cost of the current serum-free media and non-food grade compounds in these media limit the suitability of these media for cultivated meat production. Developing serum-free media for cultivated beef has been studied very well (Stout et al., 2021). In addition, one of the critical challenges in food supply chains is food loss issues that present significant sustainability and security challenges, with 60 percent of meat becoming processed waste (1.4 billion tons for livestock; 800 million tons for seafood) (Gau et al., 1999; Love et al., 2015). Oysters and mussels and their waste could be applied for protein extraction/hydrolysis for feeding the cells in the cultivated meat industry without competing with the farmed bivalves. Lugworm is also another marine invertebrate that has not been studied before for enzymatic hydrolysis. Using insects as biorefinery for converting wastes to biopolymers is also a rapidly growing industry due to the lower environmental issues compared to the conventional meat sources of protein such as beef, chicken and pork. However, one of themajor issues is the limited market for insect products, despite their high quality and nutritional value of insect protein. Therefore, using insects as a source of protein to produce the protein hydrolysates/peptides to feed the cells and reduce the serum in cell related media could be an efficient and alternative way to increase the insect protein application and production of marine-based cultivated meat.

Peptides (protein hydrolysates) from different sources, including soy, have been used for growing cells by many researchers (van der Valk et al., 2010; Kim et al., 2011; Logarusic et al., 2021; Andreassen et al., 2020) mainly for mammalian cells. However, to the best of our knowledge, no serum-free media is available for cultivated aquatic food products. Thus, developing a serum-free media using cost effective sources of growth factors and hormones will be an efficient strategy to industrialize cultivated aquatic food products. Thus, developing a serum-free media using cost effective sources of growth factors and hormones will be an efficient strategy to industrialize cultivated aquatic food products. This study aimed to develop protein hydrolysates from two insect species, including the Black soldier fly (BSF) and cricket, as well as three marine invertebrates, including oyster, mussel, and lugworm, for reducing serum in fish cell media. Moreover, we will evaluate the impact of different concentrations of protein hydrolysates on doubling time, cell biomass, and cell performance. Extracellular lactate dehydrogenase leakage (LDH) was selected as a rapid measure of cellular integrity. These rapid measurement assays can assist in screening various media formulations. For evaluating the cell performance, LDH enzyme leakage in the media will be measured to determine the membrane integrity of cells. Leaking of cell enzymes typically indicates a certain level of cytotoxicity, indicative of future apoptosis and necrosis or the initiation of tumorigenic potential (Serganova et al., 2018). During apoptosis or necrosis, the cell membrane becomes permeable, compromising its integrity, and LDH, a ubiquitous enzyme, is then released through the damaged plasma membrane and can be detected (Chan et al., 2013). This measurement enables researchers to determine if a medium allows for the proper proliferation of cells and maintains cell health over extended periods without exerting any adverse effects.

### Materials and Methods

### Materials

The BSF was provided by Chapul Farm (McMinnville, OR, USA), and the cricket powder was obtained from Cricktone (Saint Louis, Missouri, USA). Oyster, mussel, and lugworm were provided from local stores. The enzyme used in this study was commercially available-Alcalase®, an endoprotease enzyme (2.4 AU/g) from Bacillus licheniformis (Sigma-Aldrich Inc. (St. Louis, MO, USA). The zebrafish embryonic stem cell line-ZEM2S CRL-2147TM was obtained from The American Type Culture Collection (ATCC). The antibiotics which were initially used to cultivate zebrafish Embryonic Stem Cells (ESCs) were obtained from Cytiva (Marlborough, MA).

### Protein Hydrolysates Production

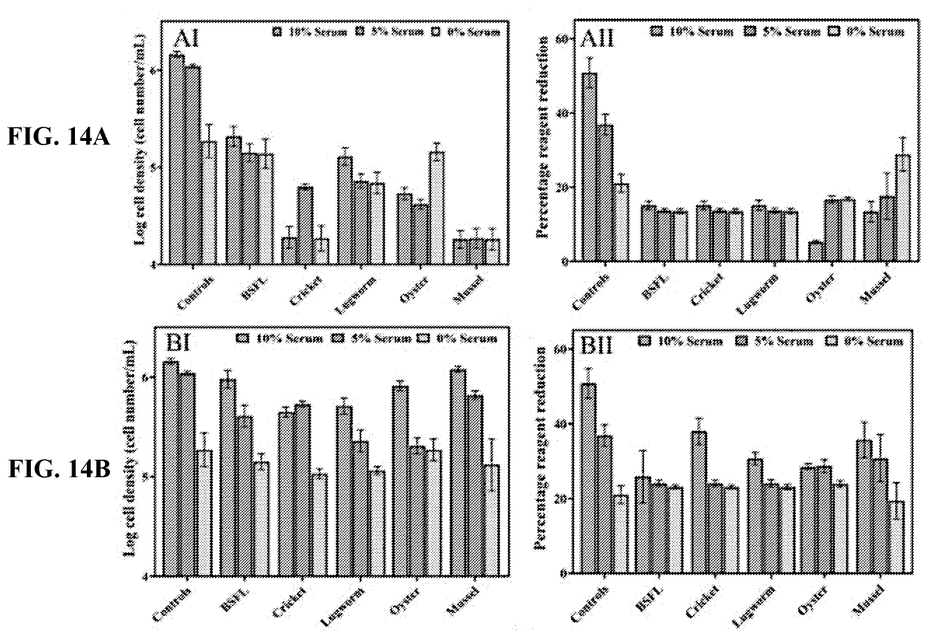

The enzymatic hydrolysis process of proteins of various sources was performed according to the previous studies (Batish et al., 2020), with slight modifications depending on the substrate (FIG. 12A). The hydrolysates were prepared in at least six replicates based on the specific hydrolysis conditions (Table 2). Briefly, raw materials were mixed with water at a specific ratio (Table 2), heated to 60° C. for 20 min followed by adding the enzyme and hydrolysis for 1 h. The enzymatic reaction was terminated by heating the samples at 90° C. for 10 min. Samples were centrifuged, and protein hydrolysates were collected and freeze-dried. The dry yield (%) and productivity (mg/mL) were determined using equations 1 and 5. Until further use, the lyophilized protein hydrolysates were kept at −20° C.

$$Yield ( \% ) = \frac{W eight of lyophilized protein hydrolysate ( g )}{W eight of raw material used ( g )} \times 100 \qquad ( Eq . 1 )$$

$$Productivity ( \frac{mg}{ml} ) = \frac{w eight of freeze dried protein hydrolysate ( mg )}{r e a ction volume ( mL )} \qquad ( Eq . 5 )$$

TABLE 2
Specific reaction conditions for enzymatic hydrolysis of substrates

| | Water to | | Hydro- | |
|---|---|---|---|---|
| | substrate | | lysis | |
| | ratio | Enzyme | time | |
| Substrate | (v/v) | (%) | (h) | Reference |
| BSF | 3:1 | 2 | 1 | (Batish et al., 2020) |
| Cricket | 3:1 | 2 | 1 | Hall et al., 2017) |
| Oyster | 3:1 | 1 | 1 | (He et al., 2020) |
| Mussel | 3:1 | 2 | 1 | (Normah & Noorasma, 2015) |
| lungworm | 1:1 | 2 | 1 | Shin et al., 2015) |

### Amino Acid Analysis, Protein Content and Degree of Hydrolysis

The total amino acid analysis was determined according to the Association of Official Agricultural Chemists (AOAC) 982.30 E (a,b,c) (AOAC, 2006). Overall, after the samples digestion with 6NHCI, ion exchange chromatography was employed with post-column ninhydrin derivatization and quantitation. The crude protein content was assessed using AOAC standard method, Kjeldahl (AOAC, 2006). Briefly, the protein content was determined by multiplying the crude nitrogen content with nitrogen to protein conversion factor (Kp), including 4.43 for BSF (Smets et al., 2021), 5 for cricket (Ritvanen et al., 2020), and 6.25 for all animal sources.

The protein quality assessment was conducted using the Digestible Indispensable Amino Acid Score (DIAAS) as recommended by FAO (FAO, 2011) using equation 6.

$$DIAAS ( \% ) = 100 \times lowest value ( \frac{mg of DIAA in 1 g of protein}{mg of DIAA in 1 g of reference protein} ) \qquad ( Eq . 6 )$$

The degree of hydrolysis was measured using indirect formol titration (Taylor 1957).

### Functional Properties

### Oil Holding Capacity (OHC)

The oil holding capacity was tested for all protein hydrolysates according to Shahidi et al. (1995) by mixing 500 mg of protein hydrolysate with 10 mL of pure canola oil. This mixture was left at room temperature for 30 minutes and gently mixed every 10 min, followed by centrifugation at 2500×g for 10 min at room temperature. The OHC was calculated according to equation 7.

$$Oil Holding Capacity = \frac{mass of sample ( with oil ) - Mass of dry}{Mass of dry sample ( g )} \qquad ( Eq . 7 )$$

### Emulsifying Capacity (EC)

The EC was evaluated by mixing 500 mg of protein hydrolysates with 50 mL of 0.1 M sodium chloride solution in a 250 ml conical flask at room temperature, according to Yasumatsu et al. (1972). The EC was calculated using equation 3.

$$Emulsyfying capacity ( EC ( \% ) ) = \frac{Total volume - A q ueous volume}{total volume} \times 100 \qquad ( Eq . 3 )$$

### Foaming Capacity (FC)

The foaming capacity was calculated according to Pacheco-Aguilar et al. (2008) by mixing 750 mg of protein hydrolysates in 25 ml distilled water for 10 min, followed by homogenization for two min. The FC was calculated using the eq. 4.

$$Foaming capacity ( FC ( \% ) ) = \frac{Aerated volume - N ormal volume}{N ormal volume} \times 100 \qquad ( Eq . 4 )$$

### Cell Culture and Maintenance

The Zebrafish embryonic stem cell (ZEMS2) was obtained from the American Type Cell Culture (ATCC). The media used to culture cells initially was Leibovitz-L-15 media (L-15), Dulbecco's Modified Eagle Media (DMEM) and Ham's F12 Media (F-12 media) in a ratio of 15:50:35 respectively, with buffering agents added 20 mM HEPES and 0.18 g/L sodium bicarbonate with 10% FBS. The cells in the ampoule were thawed at 28° C. and resuspended in 5% FBS in 9 mL media in T-25 cm2 for 30 minutes and then added up more FBS to make up 10% FBS. The subculturing process was conducted when cells reached 80-85% confluency by rinsing the cells with PBS and adding TrypLE Express to detach cells from the flask surface. The flasks were kept at 28° C. for 5 minutes to allow complete detachment, and then the cells were transferred to a 15 mL falcon tube. The TrypLE Express was neutralized by serum-free media (L-15 media) and centrifuged at 130×g for 8.5 minutes and a white pellet was obtained. The supernatant was removed, and the pellet was resuspended in 10% serum-containing media. The cell number and viability were analyzed using an automatic cell counter, and cell splitting was done based on that. The cells reached confluency in a week, with media change once on the third day.

### Cell Performance

Various protein hydrolysates were applied at different concentrations in three different experiments. In Experiment I, the impact of different concentrations of FBS on cell growth was evaluated. In Experiment II, various concentrations of protein hydrolysates (0.001, 0.01, 0.1, 1, and 10 mg/mL) in combination with media containing 0, 5, and 10% serum were studied. In experiment III, lower concentrations of protein hydrolysates (0.001, 0.01, and 0.1) (except cricket) in combination with media containing lower concentrations of serum (0, 1, 2.5, and 10%) were studied. The protein hydrolysate samples were prepared by mixing them in filter sterilized (0.22 μm) water. All the experiments were conducted in triplicate, with an initial cell density of 50,000 cells/mL. Cells were incubated for 24 hr at 28° C. in a media with 10% serum to attach the plates. The cell numbers and morphology were recorded every 24 hr and continued for three days using CKX-CCSW Confluency Checker by taking three images per well. All the hydrolysate conditions were tested in four biological replicates with three technical replicates each. The growth rate and doubling time were calculated according to equations 8 and 9.

$$growth rate ( {hr}^{- 1} ) = \frac{Ln \frac{( Viable cells at passaging )}{viable cells at seeding}}{Time ( hrs )} \qquad ( Eq . 8 )$$

$$doubling time ( hr ) = \frac{Ln ( 2 )}{growth rate ( {hr}^{- 1} )} \qquad ( Eq . 9 )$$

The viability of cells was tested by the PrestoBlue staining method. After 72 hours, the reagent 100 μL of PrestoBlue was added to the wells and incubated at 28° C. for two hr, followed by measuring the absorbances at 570 and 600 nm using a microplate reader. The dye reduction was calculated according to equation 10.

$$Reagent reduction ( \% ) = \frac{( 117216 xA 1 ) - ( 80586 XA 2 )}{( 155677 xN 2 ) - ( 1465 xN 1 )} \times 100 \qquad ( Eq . 10 )$$

where A1, A2, N1, and N2 are the absorbance of sample wells at 570 and 600 nm and media.

### Fluorescent Imaging

Optimum concentrations of protein hydrolysates were selected for imaging. The cells were fixed for 10 min with 4% paraformaldehyde and rinsed twice with PBS to remove the paraformaldehyde. The cells were made permeable to dyes by incubating them for ten min with 0.1% Tween, followed by rinsing with PBS. In order to visualize cell nuclei, 25 L of Hoechst dye was diluted with PBS at a ratio of 1:2000, added to the cells, and incubated in the dark for 10 min. After incubation, the cells were washed twice with PBS, and then 1 mL of PBS was added to each well prior to the addition of the second dye for cytoskeleton visualization. Each well was given two drops of actin green dye, incubated for 30 min, and then washed twice with PBS. The cells were then suspended in a live cell imaging solution to improve the image quality. The cells were subsequently observed using a fluorescent microscope with UV excitation/emission wavelengths of 361/486 nm and blue-cyan light with excitation/emission wavelengths of 495/518 nm.

### Lactate Dehydrogenase Activity (LDH)

The lactate dehydrogenase activity of the most potent protein hydrolysate concentrations was evaluated for 2.5 and 1% serum concentrations (only Experiment II). Cells were cultured for three days under optimum conditions. After three days, the supernatant was harvested, then transferred to 96 well plates, and the test was conducted following the instructions provided by the kit. Four biological replicates and three technical replicates per biological replicate were used for statistical reproducibility.

### Statistical Analysis

The statistical analysis was conducted using JMP 16 software. The data were tested for normality and homoscedasticity to confirm normal distribution. Normality was tested using the Shapiro-Wilk test, and Normal Quantile Plots (NQP) and the even distribution of variability was tested using Levene's test. If the data was found to be normally distributed, the One-Way ANOVA and Tukey's HSD (honestly significant difference) test was performed; otherwise, the Wilcoxson/Kruskal Wallis test and Steel Dwass/Wilcoxson pair comparisons were done with their respective controls. The differences with P<0.05 were considered statistically significant.

### Results and Discussion

### Protein Quality

The amino acid composition, protein content, and protein quality of the BSF, cricket, oyster, lugworm and mussel hydrolysates are presented in Table 3. As shown, the protein content of cricket and BSF hydrolysates is higher than lugworm, mussel and oyster hydrolysates. All protein hydrolysates contained a high concentration of essential amino acids with cell growth-promoting properties, such as alanine, glycine, proline, and aspartic acid. Protein hydrolysates were rich in glutamic acid, which also plays an essential role in animal cell culture (Ding et al., 2019; Lu & Zhao, 2018). Glutamic acid is an important source of nitrogen for de novo amino acid synthesis, which contributes to protein and nucleic acid production. In addition to providing skeletons for carbon and nitrogen biosynthesis, glutamic acid is a significant replenisher of metabolites in the tricarboxylic acid (TCA) cycle (Hosios et al., 2016). Hydrophobic amino acids such as glycine, alanine, valine, and leucine were also dominant amino acids, suggesting the presence of bioactive peptides that can have potential growth augmenting activities. All protein hydrolysates showed a high protein quality measured based on the Digestible Indispensable Amino Acid Score (DIAAS) content of essential amino acids. BSF showed the highest DIAAS score, followed by lugworm and then cricket. The lowest DIAAS score was related to mussel protein hydrolysates and then oyster. Although the cricket protein hydrolysates revealed the highest protein content, its DIAAS score was not as high as BSF due to the difference in its amino acid profile and composition.

TABLE 3
Amino acid content, protein content, and quality and
degree of hydrolysis of all hydrolysates

| Amino acid | | | | | |
|---|---|---|---|---|---|
| (g/100 g of | | | | | Lung- |
| protein) | BSL | Cricket | Oyster | Mussel | worm |
| Phenylalanine | 1.99 | 2.12 | 1.51 | 1.32 | 1.99 |
| Valine | 4.02 | 4.03 | 2.21 | 2.02 | 2.55 |
| Threonine | 2.41 | 2.91 | 1.91 | 2.04 | 2.16 |
| Tryptophan | 0.79 | 0.57 | 0.36 | 0.43 | 0.50 |
| Methionine | 0.85 | 1.25 | 1.07 | 0.88 | 1.13 |
| Leucine | 4.09 | 5.16 | 2.91 | 2.77 | 3.66 |
| Isoleucine | 2.72 | 2.84 | 1.98 | 1.84 | 2.34 |
| Lysine | 4.85 | 5.27 | 3.11 | 3.03 | 4.02 |
| Histidine | 1.85 | 1.69 | 0.83 | 0.87 | 1.14 |
| Taurine | 0.10 | 1.20 | 0.87 | 3.50 | 0.78 |
| Hydroxyproline | 0.00 | 0.20 | 0.58 | 0.28 | 0.31 |
| Aspartic Acid | 5.51 | 8.01 | 3.95 | 4.43 | 5.05 |
| Serine | 2.64 | 3.81 | 1.79 | 1.94 | 1.90 |
| Glutamic Acid | 8.51 | 10.52 | 4.81 | 5.95 | 6.71 |
| Proline | 3.94 | 3.98 | 1.60 | 1.77 | 2.06 |
| Lanthionine | 0.22 | 0.20 | 0.15 | 0.13 | 0.22 |
| Glycine | 3.23 | 4.33 | 2.09 | 3.77 | 2.70 |
| Alanine | 4.43 | 5.87 | 2.25 | 2.85 | 4.62 |
| Cysteine | 0.49 | 0.59 | 0.58 | 0.61 | 0.66 |
| Tyrosine | 3.53 | 2.65 | 1.68 | 1.48 | 1.61 |
| Hydroxylysine | 0.04 | 0.04 | 0.03 | 0.09 | 0.00 |
| Ornithine | 0.08 | 0.16 | 0.60 | 0.11 | 0.22 |
| Arginine | 3.47 | 5.76 | 2.91 | 2.82 | 3.51 |
| Total amino | 59.70 | 73.12 | 39.70 | 44.88 | 49.80 |
| acid content | | | | | |
| (g/100 g | | | | | |
| protein) | | | | | |
| Protein content | 43.95 | 69.55 | 44.69 | 54.25 | 56.59 |
| (%) | | | | | |
| DIAAS | 140.84 | 96.42 | 92.31 | 80.18 | 97.85 |
| score | | | | | |
| (%) | | | | | |
| Protein quality | Excellent | Good | Good | Good | good |

### DH and Techno-Functional Properties

The results of DH and functional properties are presented in FIG. 12B-12E. Marine invertebrates (mussels, oysters, and lugworms) showed significantly higher DH compared to the insects (BSF and cricket) (P<0.05). DH can influence the number of peptides released and their size, conformation, and amino acid sequence, which imparts various functional and bioactive properties to the hydrolysate product (Hall et al., 2017; Monaya et al., 2022). In this study, insect protein hydrolysates showed OHC values ranging between 3.54 to 4.83 g/g, which was within the previously reported range, including BSF (0.8-5.2 g/g) (Mshayisa et al., 2022; Purschke et al., 2018; Wang et al., 2021) and cricket (1.42-3.5 g/g) (Leni et al., 2020; Stone et al., 2019; Zielińska et al., 2018). Cricket had the highest OHC compared to other sources, and the mussels and oysters revealed the lowest OHC among the selected protein sources (P<0.05), which could be due to the higher content of hydrophobic amino acids in oyster, mussels and lugworm hydrolysates compared to BSF and cricket hydrolysates. To the best of our knowledge, this is the first report regarding lugworm protein hydrolysates OHC which was close to insect protein hydrolysates.

The higher OHC of the insect protein hydrolysate is potentially related to the higher content of hydrophobic amino acids in insect hydrolysates. OHC potentially can be tied to various positive attributes of protein hydrolysates that can be useful in media formulations for animal cell cultures. The culture medium comprises several hydrophobic components, such as insulin, growth factors, hormones, and carrier proteins, which are less soluble but essential for cell growth, survival, and proliferation (Bhatia et al., 2019). Due to their rigid structure and non-polar chains, lipid-based components such as sterols, cholesterol, and fatty acids are difficult to solubilize in cell media.

Lipids are essential micronutrients for cell proliferation, as these are required for membranes, signal transduction and play a role in signal transduction. Lipids are supplied exogenously as many cells cannot produce them (Achouri et al., 1998; Yao & Asayama, 2017). Hydrolyzing proteins expose their network and hydrophobic amino acids, which aid in trapping oil and potentially oil-based components, thereby boosting OHC and aiding in the solubilization and stabilization of these oil-based components (Schartl, 2014). In addition, a high oil-holding capacity is associated with increased hydrophobicity and hydrophobic amino acids, which indicates the existence of cell-proliferating bioactive peptides in the hydrolysate (Hou et al., 2017).

Mussel, oyster, and lugworm protein hydrolysates illustrated the highest foam capacity compared to insects (P<0.05) (FIG. 12B). Foaming capacity is a significant function feature for many food-based products, adding to texture or appearance. For example, bubbles define the texture of aerated commercial products, including bread, ice cream, mousse, and meringue. (Ellis & Lazidis, 2018). Foaming in a bioreactor during processing is a typical occurrence. However, it can cause significant issues, such as inhibiting cell development by limiting the surface area contact between the growth media and the bioreactor headspace and decreasing oxygen transfer rates. In addition, foam accumulation in excess can clog filters and cause vessel overpressures that exceed allowable limits. As a result, hydrolysates should not increase the foaming that occurs spontaneously in a bioreactor when added to media. Insect hydrolysates produced in this study indicated foam capacity as 4 and 20%, which is extremely low compared to the cricket and BSF found in literature which were 39-100% (Hall et al., 2017; Mshayisa et al., 2022). The bivalve protein hydrolysates showed lower foaming capacity (40-50%) than snail and scallop (51.1-160%) (Chatterjee et al., 2020; Jin et al., 2012). Foaming qualities are generally imparted by partially denaturing a globular protein as hydrophobic regions are exposed. These regions can efficiently adsorb into air-water interfaces and lower interfacial tension, thus enhancing foaming capability. However, extensive denaturation will reduce proteins' ability to produce foams (Mauer, 2003).

The results of emulsion capacity indicated that lugworm protein hydrolysates had the lowest emulsion capacity (P<0.05). Insect hydrolysates' emulsion capacity was in the range of 57.8-60%, which is in agreement with other researchers finding (39-98%) (Adebowalea et al., 2005; Mshayisa et al., 2022; Trinh & Supawong, 2021). The emulsion capacity of bivalve mollusks fell consistently in the 35-65% range as found by other studies (Haldar et al., 2018; Naik et al., 2020). An increase in emulsion capacity is usually related to the amphipathic nature of peptides that are produced by hydrolysis. It is widely reported that peptides higher than the size of 2 kDa have a higher emulsion capacity as they may possess both hydrophobic and hydrophilic amino acid chains that can interact with both water and oil-based components. However, lower emulsion capacity can be attributed to the production of extremely short peptides, which reduce the overall amphipathic nature of hydrolysate. Emulsion capacity is also another critical functional property that has industrial application when it comes to producing cultivated meat production. Culturing cells requires many water and oil-based components to ensure proper cell growth, proliferation, and sustenance, however presence of these opposite groups makes the culture media thermodynamically unstable (McClements, 2004). Emulsifying various components can help solubilize these opposite groups and allow their transfer to the cells when required. Hydrolysis of protein exposes many amphipathic, polar, and non-polar side chains that can help solubilize oil-based components and create stable emulsions so that all necessary components can be utilized by cells when required (Padial-Domínguez et al., 2020).

### Cell Morphology, Growth, and Viability

This study aimed to apply whole protein hydrolysates from different sources to reduce or replace serum only by applying protein hydrolysates without adding other growth factors.

### Impact of Serum Concentration

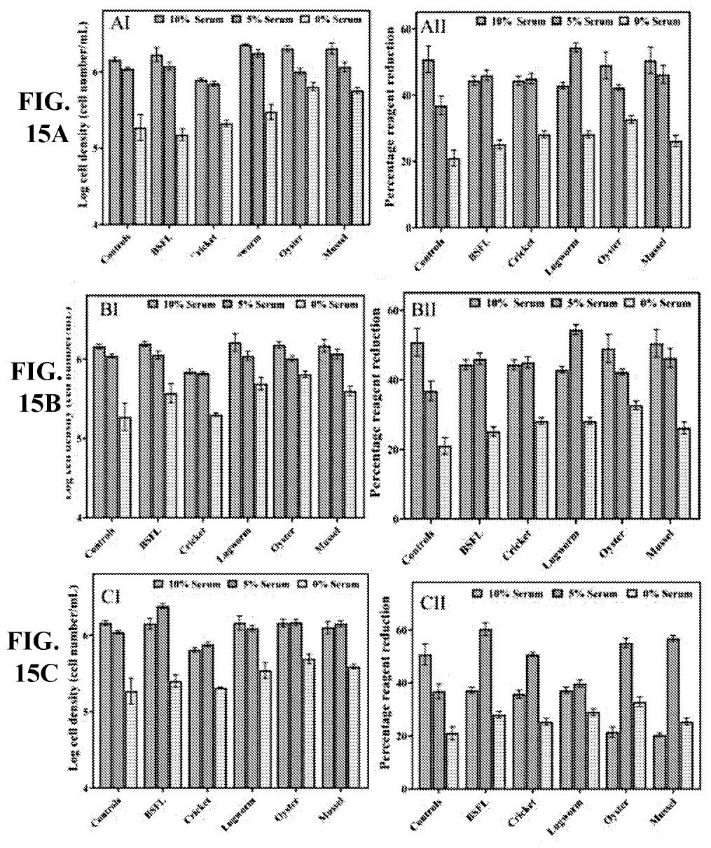

For zebrafish ESCs, 10% serum is the standard required serum concentration for regular cell growth. In the first experiment, different serum concentrations (0, 1, 2.5, 5, and 10%) were evaluated on cell growth and doubling time. The impact of serum at various concentrations on many parameters of cell growth and viability is demonstrated in FIG. 13A-13C.

The cells growth was studied daily for three days using a phase-contrast microscope equipped with an image analysis software. Direct image analysis is a dependable and accurate technique that has been demonstrated to be equivalent to other cell counting methods (Brinkmann et al., 2002; Farges-Haddani et al., 2006). After 24 hours of incubation in media containing 10% serum, the experimental media were changed to media containing 0, 1, 2.5, 5, and 10% serum. As the serum concentration decreases, the cell density reduces significantly (P<0.05) (FIG. 13A-13C). By decreasing the serum from 10% to 5, 2.5, and 1%, while cell numbers are reduced by less than 0.2 log, the cell morphology did not change, demonstrating that although cells are affected by serum reduction, they retain their original morphology (FIG. 13A). At a serum concentration of 0%, there was a significant cell reduction in the rate of cell growth and morphological changes with an increase in rounded cells. This shows that although cell growth and proliferation decreased at 2.5% serum concentration, this amount of serum was enough to maintain the normal morphology of cells. At 1% serum, the cells started showing some morphological aberrations, and severe morphological anomalies were observed with complete serum starvation. Overall, as the serum reduced, the cell death increased as cells become rounded, the cells lost their original spindle-like fibroblast morphology and became more elongated, spiky, and starved in nature which was also observed by other researchers (Pirkmajer & Chibalin, 2011). Doubling time was increased from 14.6 hr at 10% serum (Guan et al., 2019; Tamm et al., 2013; Zhan et al., 2019) to 15.54, 16.37, and 18.5 hr at 5, 2.5, and 1% serum, respectively. Doubling time was increased to 24.5 hr in serum-free media (FIG. 13C).

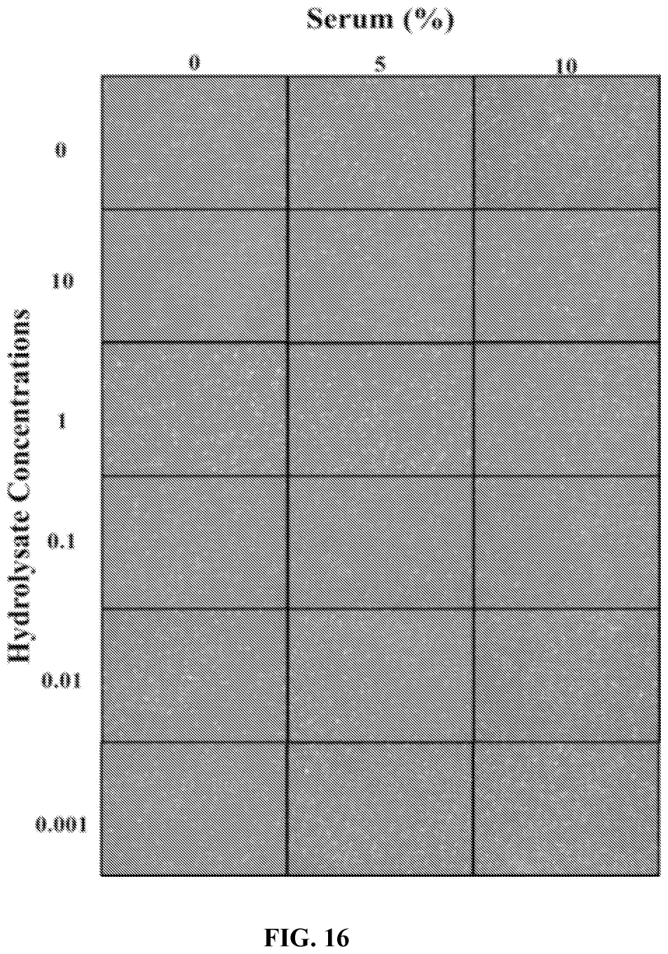

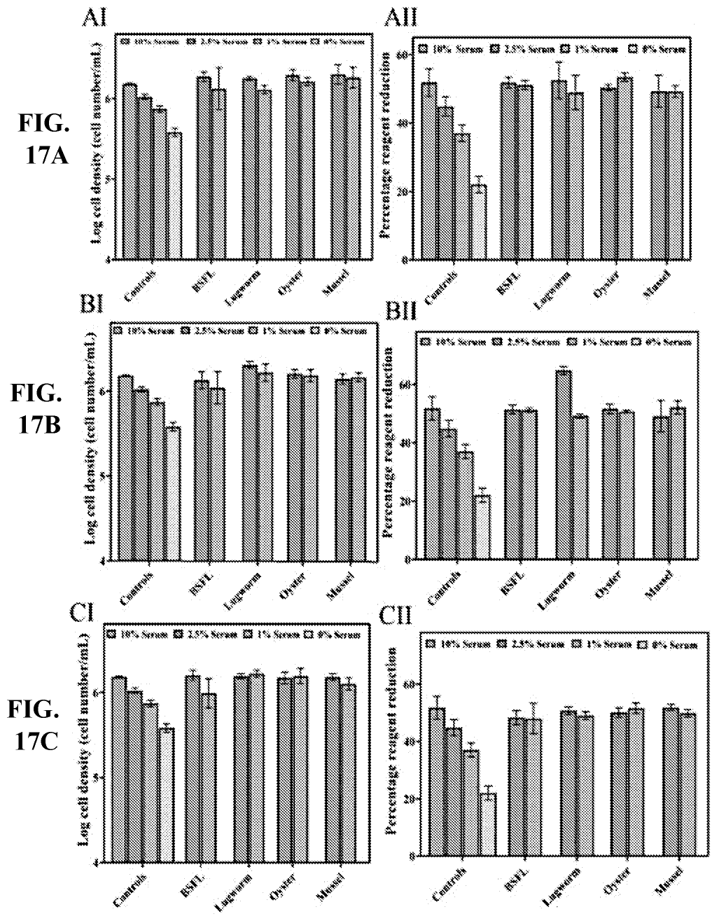

In Experiment II, we evaluated the impact of different protein hydrolysate concentrations from 0.001 to 10 mg/mL in combination with different concentrations of serum (0, 5, and 10%) on cell performance. The cell growth and cell viability results in media containing different concentrations of protein hydrolysate are presented in FIG. 14A-14B (1 and 10 mg/mL) and FIG. 15AA-15C (0.001, 0.01, and 0.1 mg/mL).

Overall, providing 1 and 10 mg/mL protein hydrolysates significantly reduced cell growth and cell viability (P<0.05). Mussel protein hydrolysates at 10 mg/mL concentrations significantly reduced cell growth compared to other hydrolysates (P<0.05). While, at 1 mg/mL concentrations, no significant differences were observed among the hydrolysates in terms of cell growth (P>0.05).

In addition, within 24 hours of adding a medium containing protein hydrolysate, the cells lost their fibroblast-like morphology, and cell death was induced. Adding these protein hydrolysate concentrations decreased the media pH despite a buffering mechanism, as the oxidation of phenol red caused the yellowing of the medium.

The lower concentrations of the lugworm, and mussel (0.01 and 0.1 mg/mL), in combination with 10% FBS, exhibited significantly higher cell proliferation compared to the control group with 10% FBS. The lowest concentration of protein hydrolysates (0.001 mg/mL) in combination with 5% serum, resulted in even higher cell growth than other tested concentrations (0.01-10 mg/mL).

At 5% serum concentration in combination with 0.01 and 0.1 mg/mL protein hydrolysates, only mussel and lugworm hydrolysates demonstrated significantly higher growth than the 10% control. While, at the lowest concentration of protein hydrolysates (0.001 mg/mL), BSF, and oyster hydrolysates, significantly increased cell growth compared to the 10% serum control group, demonstrating that depending on the type of protein hydrolysates and concentrations, supplementing the media with protein hydrolysates can reduce serum up to 50%. Using a low concentration of protein hydrolysates from lugworm, oyster, mussel, and BSF, improved cell growth significantly in a serum-free media.

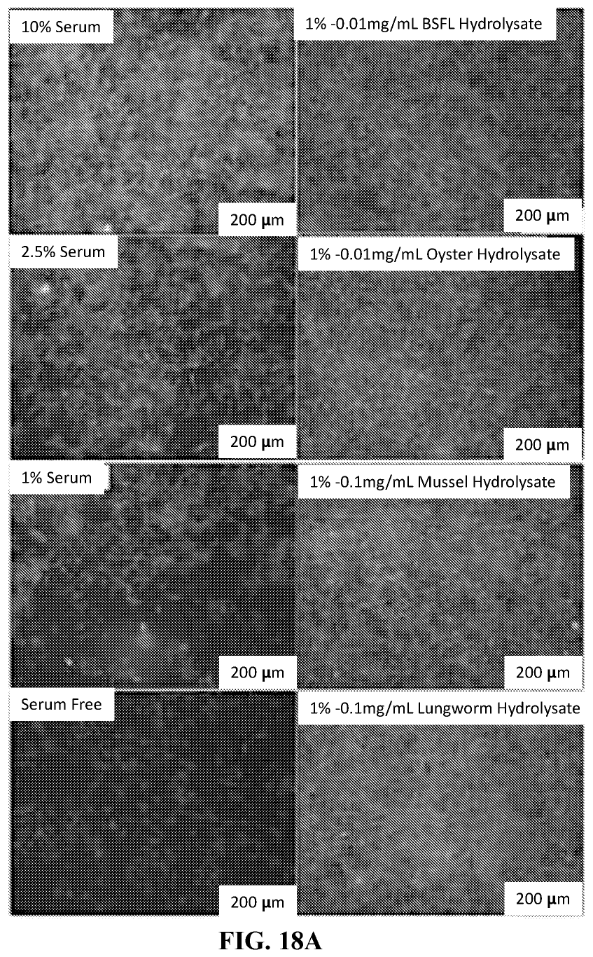

As depicted in FIG. 16, at low concentrations of protein hydrolysates (0.001, 0.01, and 0.1 mg/mL) in a serum-free media, none of the hydrolysates were able to increase the cell growth parameters as compared to the 10% serum control. However, except for cricket hydrolysates, all other hydrolysates were able to significantly increase cell growth as compared to serum-free media without protein hydrolysates (control), indicating that some protein hydrolysates could potentially support fish cell growth in a serum-free environment. While protein hydrolysates supported cell growth in serum-free media, the loss of cell morphology and starvation indicated that one source of protein hydrolysates alone could not maintain cell health, and a combination of different hydrolysates might be necessary (FIG. 16).

These results agree with other researchers findings in which a high concentration (4 to 6 mg/mL) of protein hydrolysates, including soy (Lee et al., 2008) and fish gelatin (Hsieh et al., 2022), significantly decreased cell proliferation. In addition, wheat gluten hydrolysate at concentrations between 6 and 12 mg/mL reduced cell proliferation (Radošević et al., 2016). Protein hydrolysates derived from various wastes such as eggshells and carcasses at 10 mg/mL had a similar dose-dependent, cytotoxic effect on bovine stem cells (Andreassen et al., 2020). This can be related to the presence of a high concentration of nutrients, such as oligopeptides, which disrupt the overall nutritional balance of the medium (Chun et al., 2007; Hsieh et al., 2022) and thus drastically alter its pH. In this study, growth and population doubling time were negatively impacted as a result of the death of the cells. Possibly by affecting the nutrient balance due to high amino acid or oligopeptide concentrations, as reported by (Chun et al., 2007; Hsieh et al., 2022). Lower concentrations of whey protein hydrolysate (0.01-0.5 mg/mL) boosted osteoblastic cell line proliferation, viability, and alkaline phosphatase activity (Jo et al., 2020). Similar results were observed on bovine cells with algal extract and various other industrial byproduct hydrolysates; cell growth increased relative to serum-free conditions, albeit less than in serum-rich conditions (Andreassen et al., 2020; Defendi-Cho & Gould, 2021).

The degree of hydrolysis in this study ranged from 20-32%. It has been shown that cell growth in the presence of protein hydrolysates at different concentrations of serum depends on the degree of hydrolysis (Girón-Calle et al., 2010). However, in this study, no correlation was observed between the degree of hydrolysis and cell growth. This could be explained by the fact that in this study enzymatic hydrolysis was conducted under one specific condition.

Protein hydrolysates have been demonstrated to have more than a nutritional effect on cells due to the peptides size, amino acids composition, and peptide amino acids sequences resulting in protective, anti-apoptotic, and growth-promoting properties. A study on CHO cell lines using several plant-based peptides, revealed that while protein hydrolysates did not enhance the nutritional value of the basal media, they significantly boosted cell growth (Burteau et al., 2003). Similar effects were reported when CHO cells were grown with 10% serum and algal extracts (Ng et al., 2020).

In general, all the hydrolysates, except the cricket hydrolysate, were able to reduce the serum concentration effectively and, in some cases, the cell growth was higher than the control group containing 10% serum, demonstrating the tremendous potential of these protein hydrolysates to develop serum-free media for cellular agriculture. The concentrations of 1 and 10 mg/mL appear to have a negative effect on cell growth and survival and to be possibly toxic to cell growth overall. Protein hydrolysates at lower concentrations (0.001, 0.01, and 0.1 mg/mL), did not show toxicity. Overall, the cell viability determined using PrestoBlue was comparable to results obtained by microscope image analysis which presents a good cross-reference test. The highest cell viability was related to BSL with 65% reduction.

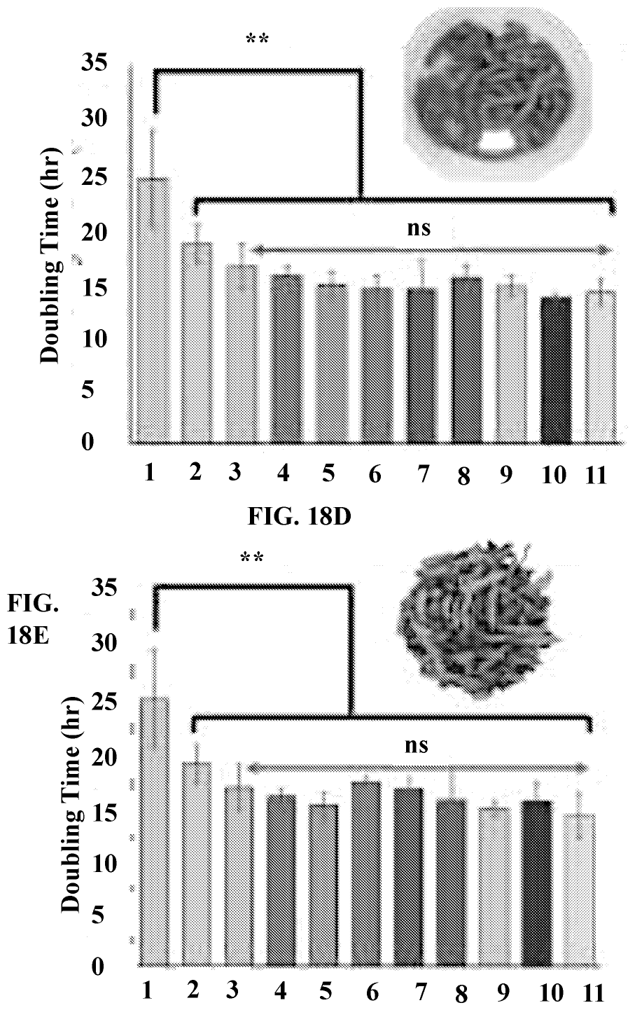

In Experiment III, lower concentrations of protein hydrolysates (0.001, 0.01, and 0.1 mg/mL) except for cricket, in combination with lower concentrations of serum (1, and 2.5%) on cell performance, were studied (FIG. 18A-18E).

All the hydrolysates at lower concentrations illustrated a positive impact on cell growth in combination with 1, and 2.5% serum (FIG. 18A-18E). Combining 0.001 mg/mL of protein hydrolysates with 1% serum increased cell biomass compared to the control groups containing 1, 2.5 and 10% serum, indicating that supplying protein hydrolysates at low concentrations can replace serum applications in cell culture media.

Most protein hydrolysates exhibited high cell growth at 2.5%, as evidenced by their growth rates and population doubling times. The cell viability profiles of all protein hydrolysates did not differ significantly from the 10% standard. Compared to the 10% serum standard, lugworm and mussel hydrolysate displayed significantly higher growth. This suggests that protein hydrolysates may synergistically affect cell growth in combination with low serum concentrations. The 0.1 mg/mL concentration efficiently reduced serum levels by up to 75% without affecting cell viability or growth profile. At 1% serum, only mussel hydrolysate exhibited significantly more growth than the control at 10% serum.

Using 0.001 mg/mL protein hydrolysates indicated that only BSF hydrolysates significantly improved the cell proliferation compared to the 10% serum control group, while other hydrolysates resulted in similar or slightly better cell proliferation compared to the 10% serum control group. Protein hydrolysate concentrations of 0.01 and 0.001 demonstrated statistically better or similar growth and viability compared to the 10% control, showing that the serum concentration can be decreased by 75 to 90% without impacting the cell performance using protein hydrolysates. Morphologically, there was no discernible difference between the protein hydrolysates at any of the serum concentrations and 10% control. This demonstrates that protein hydrolysates between 0.001-0.1 mg/mL can restore cell growth, viability parameters, and morphological integrity.

### Fluorescent Staining of Cells

Hoechst dye is an effective and reliable fluorophore with a long history of visualizing DNA content and nuclear structure using a fluorescence microscope. It is a fluorescent dye that can stain both living and fixed cells and emits fluorescence in the blue light spectrum. Serum deprivation induces apoptosis or necrosis, which can be recognized by karyopyknosis and visually identified by staining cells with hoescht dye (Bucevičius et al., 2018; Huang et al., 2018). Actin is another ubiquitous protein in animal cells, particularly in the filamentous (F) form. These proteins maintain cellular shape, structure, signaling, and cell division (Dominguez & Holmes, 2011). It is known that serum deprivation impairs actin protein, which can potentially negatively impact essential cell-based features such as cell shape, cell-cell matrix connections, and proliferative capacity (Wallenstein et al., 2010). The process of serum deprivation is poorly understood, as it differs from cell type to cell type. For this study, cells grown at all serum conditions (0-10%) and all protein hydrolysates selected from the previous section were stained with Hoescht and actin green.

From these fluorescent staining, it is evident that serum deprivation had an impact on the cytoskeleton, although there was no discernible effect on the nucleus other than a reduction in numbers. At 10% serum as control, a high number of nuclei and abundant actin protein stains were observed. At a serum concentration of 2.5%, there was no visible effect on the nucleus other than a reduction in the number. However, the actin filaments were stained less, indicating a decrease in the presence of actin proteins, and the cell area was increased. As the serum content decreased from 1 to 0%, the actin protein exhibited minimum actin staining, and the cells were more dispersed and obtained a greater surface area. Overall, the actin protein presumably decreased along with the serum concentration, which altered cell shape. This result is supported by a second investigation demonstrating a decrease in actin protein staining under serum-starvation conditions (Wallenstein et al., 2010). The selected protein hydrolysates from the first section exhibited the same number of nuclei and cytoskeleton as the 10% serum control, demonstrating that the protein hydrolysates could restore actin protein in these cells and, consequently, the original cytoskeleton.

### LDH Staining

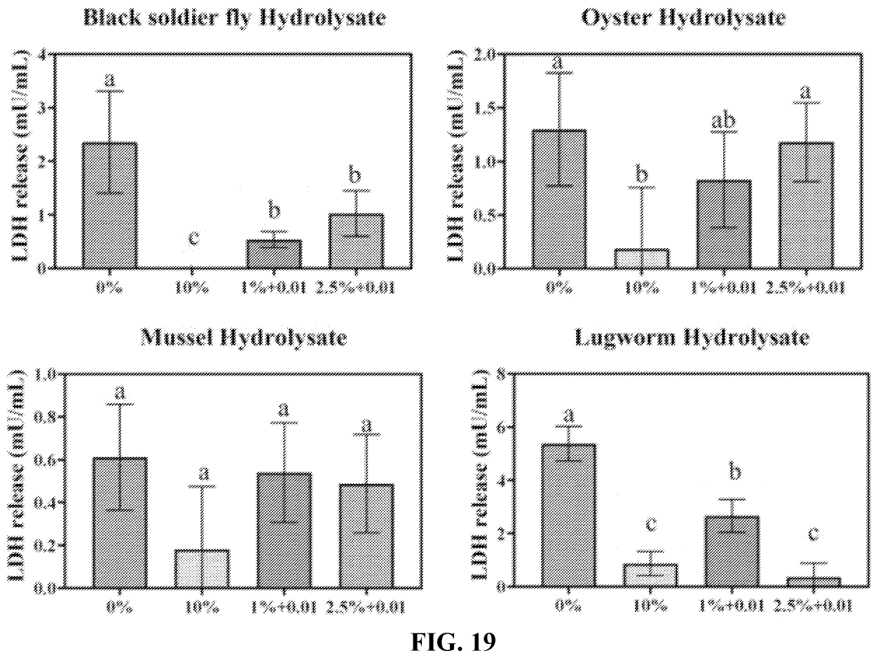

LDH activity of the selected protein hydrolysate concentration at 2.5 and 1% serum concentration and 10 and 0% control are presented in FIG. 19. In general, a serum concentration of 0% showed a more significant release of LDH, indicating damage to the plasma membrane. In contrast, a serum concentration of 10% had the lowest LDH activity, and in some cases no LDH activity was observed. These results suggest that the integrity of the cell membrane was compromised, resulting in the release of LDH, which can indicate apoptosis and necrosis. Overall, the results were highly variable due to the serum's inherent LDH activity, which can interfere with the kit. To overcome this challenge, for every study we collected specific blanks to minimize the interference from serum.

All other groups had significantly greater LDH release than the 10% control group. Only BSL, and lugworm hydrolysates significantly reduced LDH release relative to the 0% control group. All other hydrolysates exhibited a higher LDH release, indicating that these protein hydrolysates can cause a greater degree of cell membrane damage than in serum-free conditions (Chan et al., 2013).

### Yield and Cost of Protein Hydrolysates for Cultivated Meat Production

The yield, productivity and cost of production of the protein hydrolysate are provided in Table 4. For production of a hydrolysate that properly supports animal cell culture growth, the whole organism was used instead of protein isolate or concentrate. The productivity and yield of protein hydrolysates were examined to determine their potential for large-scale manufacturing. The yield and productivity values for BSF, cricket, and mussel hydrolysates were higher or comparable to those found in the literature. Cost of production for 1 kg BSF, cricket, mussel, oyster and lugworm were $91.51, $494.5, $570, $950, and $912. Based on the results from cell growth, doubling time, and cell viability, BSF and lugworm provided promising sources of protein hydrolysates for cell growth. According to this study results, the concentration of protein hydrolysate required for cultivated fish production is 0.001-0.1 mg/mL, and the yield and productivity appear suitable for cultivated meat scale up. Culturing meat requires the cultivation of billions of cells using limited space, time, and resources. Two kg batch of any protein hydrolysate would be sufficient to supply the maximum capacity of a stirred bioreactor used for animal cell culture, with a capacity of 2000 L to produce 10 to 100 kg cultivated meat (Bellani et al., 2020). Based on the economic analysis, BSF provides a more sustainable and cost-effective source of protein hydrolysates for the cultivated meat industry. First of all, BSF feeds on agri-food wastes with the conversion ratio of 2 (2 kg waste to 1 kg BSF), generates low greenhouse gas emission, produces high protein content materials, with high nutritional value (Batish et al., 2021).

TABLE 4
Dry yield and productivity (mg/mL) obtained for all protein hydrolysates

| | | | | Raw | Cost/1 | Cost for |
|---|---|---|---|---|---|---|
| | | | Yield and | material | protein | 100 kg |
| | | Productivity | productivity | cost | hydrolysates | cultivated |
| Substrate | Dry Yield (%) | (mg/mL) | from literature¹ | (per kg) | (USD)* | meat (USD)** |
| BSF | 16.57 ± 0.46 | 60.00 ± 0.00 | 10.7-6.9% 21 mg/mL | 15.16 | 91.51 | 0.915 |
| | | | 12.1-12.4% | | | |
| Cricket | 16.68 ± 1.45 | 60 ± 0.01 | 9.7-12.1% 5.7-11.2% | 82.48 | 494.5 | 4.94 |
| Mussel | 9.78 ± 0.40 | 30 ± 0.00 | 5.27-8.66% 8.34% | 55.57 | 570.1 | 5.70 |
| Oyster | 9.39 ± 0.95 | 30 ± 0.00 | — | 89.2 | 950 | 9.50 |
| Lungworm | 30.05 ± 1.24 | 100.16 ± 0.00 | — | 274.3 | 912.77 | 9.12 |

¹BSF: Abduh et al., 2020; Firmansyah and Abduh, 2019; Cricket: Hall et al., 2017; Trinh & Supawong, 2021; Mussel: Mohd Rodzi, 2015; Normah and Asmah, 2016; Oyster and lugworm: not found;
*Cost was calculated based on the yield and raw materials prices;
**Cost of required protein hydrolysates for producing 100 kg cultivated meat in a 2000 L reactor

### DISCUSSION

In this Example, the impact of different concentrations of protein hydrolysates in combination with various concentrations of serum on zebrafish embryonic stem cell performance was studied. All hydrolysates demonstrated the ability to replace at least 90% of serum in cell culture conditions, reducing the overall cost of producing cell-based meat. Although protein hydrolysates could potentially be used for developing serum-free media, more investigations are required to address the morphological changes in the cells. Based on the results of this study, the BSL hydrolysates provided optimum DH, amino acid composition, peptide size, and suitable functional properties for reduced-serum or serum-free media development. At 2.5 and 1% serum concentrations, nearly all protein hydrolysates demonstrated excellent cell growth characteristics and viability at 0.001-0.1 mg/ml concentrations, indicating that protein hydrolysates should be applied at low concentrations to support cell growth. Additionally, fluorescence imaging has shown that protein hydrolysates can improve cytoskeleton density as high as the media containing 10% serum as the control group. Lugworm and BSL hydrolysates exhibited the lowest LDH activity, indicating the least damage to the cell membrane, making them the optimum and most effective protein hydrolysate for serum-free media development without compromising cell health indicators. However, due to the cost of the protein hydrolysates, BSL provides the most cost-effective and sustainable source of protein hydrolysates for cell culture. Further investigations are required to determine the most effective peptides molecular weight and sequence on cell growth. In addition, other commercially available enzymes, and fermentation methods should be evaluated.

### REFERENCES RELATED TO EXAMPLE 3

Abduh, M. Y., Tejo, F., Hidaya, G. M. S., Eka, R. J. R., Putra, R. M., Permana, A. D., & Mandasari, M. I. (2020). Production of protein hydrolysate and biodiesel from black soldier fly larvae cultivated using rotten avocado and tofu residue. London Journal of Research in Science: Natural and Formal, 20 (8).
Achouri, A., Zhang, W., & Shiying, X. (1998). Enzymatic hydrolysis of soy protein isolate and effect of succinylation on the functional properties of resulting protein hydrolysates. Food Research International, 31 (9), 617-623.
Adebowalea, Y. A., Adebowal, K. O., & Oguntokunc, M. O. (2005). Evaluation of nutritive properties of the large African cricket (Gryllidae sp). Biological Sciences-PJSIR, 48 (4), 274-278.
Andreassen, R. C., Pedersen, M. E., Kristoffersen, K. A., & Rønning, S. B. (2020). Screening of by-products from the food industry as growth promoting agents in serum-free media for skeletal muscle cell culture. Food & Function, 11 (3), 2477-2488.
AOAC. (2006). Method 982.30, Official Methods of Analysis. AOAC International Gaithersburg, MD.
Batish, I., Brits, D., Valencia, P., Miyai, C., Rafeeq, S., Xu, Y., Galanopoulos, M., Sismour, E., & Ovissipour, R. (2020). Effects of Enzymatic Hydrolysis on the Functional Properties, Antioxidant Activity and Protein Structure of Black Soldier Fly (Hermetia illucens) Protein. Insects, 11 (12), 876. https://doi.org/10.3390/insects11120876.
Bellani, C. F., Ajeian, J., Duffy, L., Miotto, M., Groenewegen, L., & Connon, C. J. (2020). Scale-Up Technologies for the Manufacture of Adherent Cells. Frontiers in Nutrition, 7, 178. https://doi.org/10.3389/fnut.2020.575146
Bhatia, S., Naved, T., & Sardana, S. (2019). Introduction to Pharmaceutical Biotechnology, Volume 3. In Animal tissue culture and biopharmaceuticals. IOP Publishing. https://doi.org/10.1088/2053-2563/aafac0
Brinkmann, M., Lütkemeyer, D., Gudermann, F., & Lehmann, J. (2002). New technologies for automated cell counting based on optical image analysisThe Cellscreen′. Cytotechnology, 38 (1), 119-127.
Bucevičius, J., Lukinavičius, G., & Gerasimaitė, R. (2018). The Use of Hoechst Dyes for DNA Staining and Beyond. In Chemosensors (Vol. 6, Issue 2). https://doi.org/10.3390/chemosensors6020018
Burteau, C. C., Verhoeye, F. R., Molsl, J. F., Ballez, J.-S., Agathos, S. N., & Schneider, Y.-J. (2003). Fortification of a protein-free cell culture medium with plant peptones improves cultivation and productivity of an interferon-γ-producing CHO cell line. In Vitro Cellular & Developmental Biology-Animal, 39 (7), 291-296.
Chan, F. K.-M., Moriwaki, K., & De Rosa, M. J. (2013). Detection of necrosis by release of lactate dehydrogenase activity. Methods in Molecular Biology (Clifton, N.J.), 979, 65-70. https://doi.org/10.1007/978-1-62703-290-2 7
Chatterjee, R., Dey, T. K., Roychoudhury, A., Paul, D., & Dhar, P. (2020). Enzymatically excised oligopeptides from Bellamya bengalensis shows potent antioxidative and anti-hypertensive activity. Journal of Food Science and Technology, 57 (7), 2586-2601.
Chun B H, Kim J H, Lee H J, Chung NH (2007) Usability of size excluded fractions of soy protein hydrolysates for growth and viability of Chinese hamster ovary cells in protein-free suspension culture. Bioresour Technol 98:1000-1005
Defendi-Cho, G., & Gould, T. M. (2021). In Vitro Culture of Bovine Fibroblasts using Select Serum-Free Media Supplemented with Chlorella vulgaris Extract. BioRxiv.
Ding, D., Yu, T., Du, B., & Huang, Y. (2019). Collagen hydrolysate from Thunnus orientalis bone induces osteoblast proliferation and differentiation. Chemical Engineering Science, 205, 143-150.
Dominguez, R., & Holmes, K. C. (2011). Actin structure and function. Annual Review of Biophysics, 40, 169-186. https://doi.org/10.1146/annurev-biophys-042910-155359
Ellis, A., & Lazidis, A. (2018). Foams for Food Applications. In Polymers for Food Applications (pp. 271-327). https://doi.org/10.1007/978-3-319-94625-2 11
FAO. (2011). Dietary protein quality evaluation in human nutrition. FAO Food Nutr. Pap, 92, 1-66.
FAO. (2020). The State of World Fisheries and Aquaculture 2020. FAO. https://doi.org/10.4060/ca9229en
Farges-Haddani, B., Tessier, B., Chenu, S., Chevalot, I., Harscoat, C., Marc, I., Goergen, J. L., & Marc, A. (2006). Peptide fractions of rapeseed hydrolysates as an alternative to animal proteins in CHO cell culture media. Process Biochemistry, 41 (11), 2297-2304.
Firmansyah, M., & Abduh, M. Y. (2019). Production of protein hydrolysate containing antioxidant activity from Hermetia illucens. Heliyon, 5 (6), e02005.
GAO U. Animal Agriculture: Waste Management Practices. Report to the Honorable Tom Harkin, Ranking Minority Member, Committee on Agriculture, Nutrition, and Forestry, US SenateUS General Accounting Office. 1999.
Girón-Calle J, Alaiz M, Vioque J. Effect of chickpea protein hydrolysates on cell proliferation and in vitro bioavailability. Food Research International. 2010-6-1;43 (5): 1365-70.
Guan, Y., Xie, Y., Li, D., Zhu, Y., Zhang, X., Feng, Y., Chen, Y., Xu, L., Liao, P., & Wang, G. (2019). Comparison of biological characteristics of mesenchymal stem cells derived from the human umbilical cord and decidua parietalis. Molecular Medicine Reports, 20 (1), 633-639.
Haldar, A., Das, M., Chatterjee, R., Dey, T. K., Dhar, P., & Chakrabarti, J. (2018). Functional properties of protein hydrolysates from fresh water mussel Lamellidens marginalis (Lam.).
Hall, F. G., Jones, O. G., O'Haire, M. E., & Liceaga, A. M. (2017). Functional properties of tropical banded cricket (Gryllodes sigillatus) protein hydrolysates. Food Chemistry, 224, 414-422.
He, S., Chen, Y., Brennan, C., Young, D. J., Chang, K., Wadewitz, P., Zeng, Q., & Yuan, Y. (2020). Antioxidative activity of oyster protein hydrolysates Maillard reaction products. Food Science & Nutrition, 8 (7), 3274-3286.
Hosios, A. M., Hecht, V. C., Danai, L. V, Johnson, M. O., Rathmell, J. C., Steinhauser, M. L., Manalis, S. R., & Vander Heiden, M. G. (2016). Amino acids rather than glucose account for the majority of cell mass in proliferating mammalian cells. Developmental Cell, 36 (5), 540-549.
Hou, Y., Wu, Z., Dai, Z., Wang, G., & Wu, G. (2017). Protein hydrolysates in animal nutrition: Industrial production, bioactive peptides, and functional significance. Journal of Animal Science and Biotechnology, 8 (1), 24.
Hsieh, C.-H., Wang, T.-Y., Tung, B.-C., Liu, H.-P., Yeh, L.-T., & Hsu, K.-C. (2022). The Hydrolytic Peptides of Soybean Protein Induce Cell Cycle Arrest and Apoptosis on Human Oral Cancer Cell Line HSC-3. Molecules, 27 (9), 2839.
Huang, Y., Fu, Z., Dong, W., Zhang, Z., Mu, J., & Zhang, J. (2018). Serum starvation-inducesdown-regulation of Bcl-2/Bax confers apoptosis in tongue coating-related cells in vitro. Molecular Medicine Reports, 17 (4), 5057-5064. https://doi.org/10.3892/mmr.2018.8512
Jin, W.-G., Wu, H.-T., Zhu, B.-W., & Ran, X.-Q. (2012). Functional properties of gelation-like protein hydrolysates from scallop (Patinopecten yessoensis) male gonad. European Food Research and Technology, 234 (5), 863-872.
Jo, K., Hong, K.-B., & Suh, H. J. (2020). Effects of the Whey Protein Hydrolysates of Various Protein Enzymes on the Proliferation and Differentiation of 3T3-E1 Osteoblasts. Preventive Nutrition and Food Science, 25 (1), 71-77. https://doi.org/10.3746/pnf.2020.25.1.71
Kim, J. Y., Kim, Y.-G., Han, Y. K., Choi, H. S., Kim, Y. H., & Lee, G. M. (2011). Proteomic understanding of intracellular responses of recombinant Chinese hamster ovary cells cultivated in serum-free medium supplemented with hydrolysates. Applied Microbiology and Biotechnology, 89 (6), 1917-1928.
Lee, Y. K., Kim, S. Y., Kim, K. H., Chun, B.-H., Lee, K.-H., Oh, D. J., & Chung, N. (2008). Use of soybean protein hydrolysates for promoting proliferation of human keratinocytes in serum free medium. Biotechnology Letters, 30 (11), 1931-1936.
Leni, G., Soetemans, L., Caligiani, A., Sforza, S., & Bastiaens, L. (2020).Degree of Hydrolysis Affects the Techno-Functional Properties of Lesser Mealworm Protein Hydrolysates. Foods, 9 (4), 381.
Logarušić, M., Gaurina Srček, V., Berljavac, S., Leboš Pavunc, A., Radošević, K., & Slivac, I. (2021). Protein Hydrolysates from Flaxseed Oil Cake as a Media Supplement in CHO Cell Culture. In Resources (Vol. 10, Issue 6). https://doi.org/10.3390/resources10060059.
Love DC, Fry JP, Milli MC, Neff RA. Wasted seafood in the United States: Quantifying loss from production to consumption and moving toward solutions. Global Environmental Change. 2015-11-1; 35:116-24.
Lu, M., & Zhao, X.-H. (2018). The growth proliferation, apoptotic prevention, and differentiation induction of the gelatin hydrolysates from three sources to human fetal osteoblasts (hFOB 1.19 cells). Molecules, 23 (6), 1287.
Mauer, L. (2003). PROTEIN| Heat Treatment for Food Proteins (B. B. T.-E. of F. S. and N. (Second E. Caballero (ed.); pp. 4868-4872). Academic Press. https://doi.org/https://doi.org/10.1016/B0-12-227055-X/00988-3
McClements, D. J. (2004). Food emulsions: principles, practices, and techniques. CRC press.
Mohd Rodzi, N. D. (2015). Production of green mussel hydrolysate (Perna viridis) for umami flavor development. Universiti Teknologi MARA.
Monaya, K. J. M., Gomez, H. L. R., & Tejano, L. A. (2022). Production and Characterization of Protein Isolates and Hydrolysates from Slipper Cupped Oyster (Crassostrea iredalei). Philippine Journal of Science, 151 (3), 853-861.
Mshayisa, V. V., Van Wyk, J., & Zozo, B. (2022). Nutritional, Techno-Functional and Structural Properties of Black Soldier Fly (Hermetia illucens) Larvae Flours and Protein Concentrates. Foods, 11 (5), 724.
Naik, A. S., Mora, L., & Hayes, M. (2020). Characterisation of seasonal Mytilus edulis byproducts and generation of bioactive hydrolysates. Applied Sciences, 10 (19), 6892.
Ng, J. Y., Chua, M. L., Zhang, C., Hong, S., Kumar, Y., Gokhale, R., & Ee, P. L. R. (2020). Chlorella vulgaris extract as a serum replacement that enhances mammalian cell growth and protein expression. Frontiers in Bioengineering and Biotechnology, 8, 1068.
Normah, I., & Noorasma, M. (2015). Physicochemical properties of mud clam (Polymesoda erosa) hydrolysates obtained using different microbial enzymes.

Normah, I., & Asmah, A. H. (2016). Characterization of green mussel (Perna viridis) hydrolysate prepared using alcalase and starfruit (Averrhoa carambola. L) protease. International Food
Research Journal, 23 (4), 1409. Pacheco-Aguilar, R., Mazorra-Manzano, M. A., & Ramírez-Suárez, J. C. (2008). Functional properties of fish protein hydrolysates from Pacific whiting (Merluccius productus) muscle produced by a commercial protease. Food Chemistry, 109 (4), 782-789. https://doi.org/10.1016/j.foodchem.2008.01.047
Padial-Domínguez, M., Espejo-Carpio, F. J., Pérez-Gálvez, R., Guadix, A., & Guadix, E. M. (2020). Optimization of the emulsifying properties of food protein hydrolysates for theproduction of fish oil-in-water emulsions. Foods, 9 (5), 636.
Pirkmajer, S., & Chibalin, A. V. (2011). Serum starvation: caveat emptor. American Journal of Physiology-Cell Physiology, 301 (2), C272-C279.
Purschke, B., Meinlschmidt, P., Horn, C., Rieder, O., & Jäger, H. (2018). Improvement of technofunctional properties of edible insect protein from migratory locust by enzymatic hydrolysis. European Food Research and Technology, 244 (6), 999-1013.
Radošević, K., Dukić, B., Andlar, M., Slivac, I., & Srček, V. G. (2016). Adaptation and cultivation of permanent fish cell line CCO in serum-free medium and influence of protein hydrolysates on growth performance. Cytotechnology, 68 (1), 115-121.
Rashid, M., & Coombs, K. M. (2019). Serum-reduced media impacts on cell viability and protein expression in human lung epithelial cells. Journal of Cellular Physiology, 234 (6), 7718-7724.
Ritvanen T, Pastell H, Welling A, Raatikainen M. The nitrogen-to-protein conversion factor of two cricket species-Achetadomesticus and Gryllus bimaculatus. Agric Food Sci. 2020; 29 (1): 1-5.
Schartl, M. (2014). Beyond the zebrafish: diverse fish species for modeling human disease. Disease Models &amp;Amp; Mechanisms, 7 (2), 181 LP-192. https://doi.org/10.1242/dmm.012245
Serganova, I., Cohen, I. J., Vemuri, K., Shindo, M., Maeda, M., Mane, M., Moroz, E., Khanin, R., Satagopan, J., Koutcher, J. A., & Blasberg, R. (2018). LDH-A regulates the tumor
microenvironment via HIF-signaling and modulates the immune response. PloS One, 13 (9), e0203965. https://doi.org/10.1371/journal.pone.0203965
Shahidi, F., Han, X.-Q., & Synowiecki, J. (1995). Production and characteristics of protein hydrolysates from capelin (Mallotus villosus). Food Chemistry, 53 (3), 285-293.
Shin, J. E., Kang, S. M., Koh, J., & Kwon, Y. J. (2015). Effect of lugworm protein hydrolysates containing proteins of differing molecular weight distributions on permanent wave treatment in damaged hair. Materials Research Innovations, 19 (sup5), S5-1009.
Smets R, Claes J, Van Der Borght M. On the nitrogen content and a robust nitrogen-to-protein conversion factor of black soldier fly larvae (Hermetia illucens). Analytical and Bioanalytical Chemistry. 2021 October;413 (25): 6365-77.
Stone, A. K., Tanaka, T., & Nickerson, M. T. (2019). Protein quality and physicochemical properties of commercial cricket and mealworm powders. Journal of Food Science and Technology, 56 (7), 3355-3363.
Stout, A. J., Mirliani, A. B., White, E. C., Yuen, J. S. K., & Kaplan, D. L. (2021). Simple and effective serum-free medium for sustained expansion of bovine satellite cells for cell cultured meat. BioRxiv.
Tamm, C., Pijuan Galitó, S., & Annerén, C. (2013). A comparative study of protocols for mouse embryonic stem cell culturing. PloS One, 8 (12), e81156.
Taylor W H. Formol titration: an evaluation of its various modifications. Analyst. 1957-1-1;82 (976): 488-98.
Terra, L. F., Garay-Malpartida, M. H., Wailemann, R. A. M., Sogayar, M. C., & Labriola, L. (2011). Recombinant human prolactin promotes human beta cell survival via inhibition of extrinsic and intrinsic apoptosis pathways. Diabetologia, 54 (6), 1388-1397.
Trinh, B. T., & Supawong, S. (2021). Enzymatic hydrolysis of cricket (Gryllodes sigillatus) protein: Influence of Alcalase and Neutrase enzyme on functional properties of recovered protein. Thai Journal of Science and Technology, 10 (3), 342-353.
van der Valk, J., Brunner, D., De Smet, K., Fex Svenningsen, Å., Honegger, P., Knudsen, L. E., Lindl, T., Noraberg, J., Price, A., Scarino, M. L., & Gstraunthaler, G. (2010). Optimization of chemically defined cell culture media-Replacing fetal bovine serum in mammalian in vitro methods. Toxicology in Vitro, 24 (4), 1053-1063. https://doi.org/10.1016/j.tiv.2010.03.016
Wallenstein, E. J., Barminko, J., Schloss, R. S., & Yarmush, M. L. (2010). Serum starvation improves transient transfection efficiency in differentiating embryonic stem cells. Biotechnology Progress, 26 (6), 1714-1723.
Wang, J., Jousse, M., Jayakumar, J., Fernández-Arteaga, A., de Lamo-Castellvi, S., Ferrando, M., & Güell, C. (2021). Black Soldier Fly (Hermetia illucens) Protein Concentrates as a Sustainable Source to Stabilize O/W Emulsions Produced by a Low-Energy High-Throughput Emulsification Technology. Foods, 10 (5), 1048.
Yao, T., & Asayama, Y. (2017). Animal-cell culture media: history, characteristics, and current issues. Reproductive Medicine and Biology, 16 (2), 99-117.
Yasumatsu, K., Sawada, K., Moritaka, S., Misaki, M., Toda, J., Wada, T., & Ishii, K. (1972). Whipping and emulsifying properties of soybean products. Agricultural and Biological Chemistry, 36 (5), 719-727.
Zhan, X.-S., El-Ashram, S., Luo, D.-Z., Luo, H.-N., Wang, B.-Y., Chen, S.-F., Bai, Y.-S., Chen, Z.-S., Liu, C.-Y., & Ji, H.-Q. (2019). A comparative study of biological characteristics and transcriptome profiles of mesenchymal stem cells from different canine tissues. International Journal of Molecular Sciences, 20 (6), 1485.
Zhang Y, Zhou Y, Yu J (1994) Effects of peptone on hybridoma growth and monoclonal antibody formation. Cytotechnology 16:147-150
Zielińska, E., Karaś, M., & Baraniak, B. (2018). Comparison of functional properties of edible insects and protein preparations thereof. Lwt, 91, 168-174.

***

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. A composition comprising: an amount of an algae protein hydrolysate, an amount of a plant protein hydrolysate, an amount of a fungi protein hydrolysate, an amount of an insect protein hydrolysate, an amount of a multicellular marine organism protein hydrolysate, or any combination thereof.
2. The composition of aspect 1, wherein the composition is a cell culture media supplement, a partially complete cell culture media, or a complete cell culture media.
3. The composition of aspect 1, wherein the composition is a serum replacer.
4. The composition of any one of aspects 1-3, wherein the algae protein hydrolysate is prepared from: Chlorophyta (green algae), Rhodophyta (red algae), Stramenopiles (heterokonts), Xanthophyceae (yellow-green algae), Glaucocystophyceae (glaucocystophytes), Chlorarachniophyceae (chlorarachniophytes), Euglenida (euglenids), Haptophyceae (coccolithophorids), Chrysophyceae (golden algae), Cryptophyta (cryptomonads), Dinophyceae (dinoflagellates), Haptophyceae (coccolithophorids), Bacillariophyta (diatoms), Eustigmatophyceae (eustigmatophytes), Raphidophyceae (raphidophytes), Scenedesmaceae and Phaeophyceae (brown algae), or any combination thereof.
5. The composition of any one of aspects 1-4, wherein the algae protein hydrolysate is prepared from: Chlamydomonas reinhardtii, Dunaliella salina, Haematococcus pluvialis, Chlorella vulgaris, Acutodesmus obliquus, Scenedesmus dimorphus, and any combination thereof.
6. The composition of any one of aspects 1-4, wherein the algae protein hydrolysate is prepared from: Chlamydomonas, Dunaliella, Haematococcus, Chlorella, Scenedesmaceae, and any combination thereof.
7. The composition of any one of aspects 1-4, wherein the algae protein hydrolysate is prepared from:
   a. the Chlamydomonas is a Chlamydomonas reinhardtii,
   b. the Chlorella is a Chlorella minutissima or a Chlorella sorokiniana, or
   c. both (a) and (b).
8. The composition of any one of aspects 1-4, wherein the algae protein hydrolysate is prepared from: a Rodophyceae (red seaweed), optionally a Gigartinaceae or a Soliericeae.
9. The composition of aspect 8, wherein the Rodophyceae is Chondrus crispus, Chondrus ocellatus, Eucheuma cottonii, Eucheuma spinosum, Gigartina acicularis, Gigartina pistillata, Gigartina radula, Gigartina stellate, Furcellaria fastigiata, Analipus japonicus, Eisenia bicyclis, Hizikia fusiforme, Kjellmaniella gyrata, Laminaria angustata, Laminaria longirruris, Laminaria Longissima, Laminaria ochotensis, Laminaria claustonia, Laminaria saccharina, Laminaria digitata, Laminaria japonica, Macrocystis pyrifera, Petalonia fascia, Scytosiphon lome, Gloiopeltis furcata, Porphyra crispata, Porhyra deutata, Porhyra perforata, Porhyra suborbiculata, Porphyra tenera, Rhodymenis palmate, or any combination thereof.
10. The composition of any one of aspects 1-9, wherein the plant protein hydrolysate is prepared from: Pea (Pisum sativum), Sorghum (Sorghum bicolor), rice (Oryza sativa), wheat (Triticum), Hemp (Cannabis sativa), quinoa (Chenopodium quinoa), soybean (Glycine max), Corn, beans family, Chickpeas, Lentils, Peanuts, Almonds, Chia seeds, Potatoes, Seitan, oat, Almond, barley, Brewers Spent Grain, or any combination thereof.
11. The composition of any one of aspects 1-10, wherein the fungi protein hydrolysate is prepared from yeast.
12. The composition of aspect 11, wherein the yeast is Saccharomyces sp., Candida utilis, Lipomyces starkeyi and Phaffia rhodozyma, Fusarium moniliforme, Rhizopus niveus, Rhizopus oryzae, Aspergillus niger, Aspergillus oryzae, Candida guilliermondii, Candida lipolytica, Candida pseudotropicalis, Mucor pusillus Lindt, Mucor miehei, Rhizomucor miehei, Morteirella vinaceae, Endothia parasitica, Kluyveromyces lactis (previously called Saccharomyces lactis), Kluyveromyces marxianus, Lipomyces starkeyi, Rhodotorula colostri, Rhodotorula dairenensis, Rhodotorula glutinis, Rhodosporium diobovatum, Schizosaccharomyces pombe, Eremothecium ashbyii; Microfungi, optionally Fusarium venenatum, mushrooms, optionally Agaricus bisporus, Agaricus, Shiitake, Pleurotus ostreatus (oyster mushroom), Tremella fuciformis, Pleurotus eryngii, and/or Enoki, or any combination thereof.
13. The composition of any one of aspects 1-12, wherein the insect protein hydrolysate is prepared from Hermetia illucens, Galleria mellonella, Acheta domesticus, Dytiscus marginalis, Lethocerus indicus, Locusta migratoria, Gryllotalpa Gryllotalpa, Bombyx mori, Rhynchophorus ferrugineus, Blatta orientalis, Mesobuthus martensii, Gryllus bimaculatus, Acheta domesticus, Gryllus assimilis, Tenebrio molitor, Cicadidae, or any combination thereof.
14. The composition of any one of aspects 1-13, wherein the multicellular marine organism protein hydrolysate is prepared from Arenicola marina, enchytraeus albidusm, Chironomidae, Hirudinea, Eisenia fetida, Perionyx excavates, Gastropoda, Mytilus edulis, Mytilus californianus, Crassostrea virginica, Crassostrea gigas, Stomolophus Meleagris, Lycastopsis catarractarum, Glycera, Nereidae, Artemia sp., Rotifera sp., Copepoda, Mercenaria mercenaria, Corbicula manilensis, Daphnia sp. Euphausiacea, Gammarus, or any combination thereof.
15. The composition of any one of aspects 1-14, wherein the amount of each of the protein hydrolysates present in the composition ranges from any non-zero number greater than 0 to 100%.
16. The composition of any one of aspects 1-15, wherein each hydrolysate present in the composition ranges in concentration as based on the total composition any non-zero concentration greater than 0 mg/mL to 100 mg/mL.
17. The composition of any one of aspects 1-16, wherein the average peptide size of the composition ranges from 2-50 amino acids.
18. The composition of any one of aspects 1-17, wherein the degree of hydrolysis for any one or more of the hydrolysates independently ranges from 0 to 100%.
19. The composition of any one of aspects 1-18, further comprising one or more nutrients, one or more pH indicators, one or more antibiotics, one or more antifungals, one or more biologic factors or agents, one or more buffering agents, one or more salts, one or more co-factors, one or more trace elements, a cell viability colorimetric agent, or any combination thereof.
20. The composition of any one of aspects 1-19, further comprising one or more cells.
21. The composition of aspect 20, wherein the one or more cells are eukaryotic cells.
22. A method of cell culture comprising: culturing one or more cells in a composition of any one of aspects 1-21.
23. The method of aspect 22, wherein culturing comprises, plating, growing, passaging, expanding, splitting, or any combination thereof, one or more times.
24. A method of preparing a composition of any one of aspects 1-21, the method comprising: blending one or more protein sources with water at a ratio of protein sources to water to form a slurry; adding alcalase to the slurry formed in (a) for a period of time to form a hydrolysate and one or more byproducts; inactivating enzymes present in the hydrolysate and the one or more byproducts; separating the hydrolysate from the one or more byproducts; and optionally storing and/or retaining at least the hydrolysate.
25. The method of aspect 24, wherein (b) is performed at about 60 degrees C.
26. The method of any one of aspects 24-25, wherein (b) is performed at a pH of about 6-8
27. The method of any one of aspects 24-26, wherein (b) is performed with agitation.
28. The method of any one of aspects 24-27, wherein the one or more byproducts are oil, sludge, or both.
29. The method of any one of aspects 24-28, wherein (d) separating comprises centrifuging the hydrolysate and the one or more byproducts.
30. The method of any one of aspects 24-29, wherein the one or more protein sources are selected from a plant, a fungi, an algae, an insect, a multicellular marine organism, or any combination thereof.
31. The method of aspect 30, wherein the plant is Pea (Pisum sativum), Sorghum (Sorghum bicolor), rice (Oryza sativa), wheat (Triticum), Hemp (Cannabis sativa), quinoa (Chenopodium quinoa), soybean (Glycine max), Corn, beans family, Chickpeas, Lentils, Peanuts, Almonds, Chia seeds, Potatoes, Seitan, oat, Almond, barley, Brewers Spent Grain, or any combination thereof.
32. The method of any one of aspects 30-31, wherein the algae is Chlorophyta (green algae), Rhodophyta (red algae), Stramenopiles (heterokonts), Xanthophyceae (yellow-green algae), Glaucocystophyceae (glaucocystophytes), Chlorarachniophyceae (chlorarachniophytes), Euglenida (euglenids), Haptophyceae (coccolithophorids), Chrysophyceae (golden algae), Cryptophyta (cryptomonads), Dinophyceae (dinoflagellates), Haptophyceae (coccolithophorids), Bacillariophyta (diatoms), Eustigmatophyceae (eustigmatophytes), Raphidophyceae (raphidophytes), Scenedesmaceae and Phaeophyceae (brown algae), or any combination thereof.
33. The method of any one of aspects 30-32, wherein algae is selected from the group consisting of: Chlamydomonas reinhardtii, Dunaliella salina, Haematococcus pluvialis, Chlorella vulgaris, Acutodesmus obliquus, Scenedesmus dimorphus, and any combination thereof.
34. The method of any one of aspects 32-33, wherein the green algae is selected from the group consisting of: Chlamydomonas, Dunaliella, Haematococcus, Chlorella, Scenedesmaceae, and any combination thereof.
35. The method of aspect 34, wherein
   a. the Chlamydomonas is a Chlamydomonas reinhardtii,
   b. the Chlorella is a Chlorella minutissima or a Chlorella sorokiniana, or
   c. both (a) and (b).
36. The method of any one of aspects 30-35, wherein the algae is a Rodophyceae (red seaweed), optionally a Gigartinaceae or a Soliericeae.
37. The method of aspect 36, wherein the Rodophyceae is Chondrus crispus, Chondrus ocellatus, Eucheuma cottonii, Eucheuma spinosum, Gigartina acicularis, Gigartina pistillata, Gigartina radula, Gigartina stellate, Furcellaria fastigiata, Analipus japonicus, Eisenia bicyclis, Hizikia fusiforme, Kjellmaniella gyrata, Laminaria angustata, Laminaria longirruris, Laminaria Longissima, Laminaria ochotensis, Laminaria claustonia, Laminaria saccharina, Laminaria digitata, Laminaria japonica, Macrocystis pyrifera, Petalonia fascia, Scytosiphon lome, Gloiopeltis furcata, Porphyra crispata, Porhyra deutata, Porhyra perforata, Porhyra suborbiculata, Porphyra tenera, Rhodymenis palmate, or any combination thereof.
38. The method of any one of aspects 30-37, wherein the insect is Hermetia illucens, Galleria mellonella, Acheta domesticus, Dytiscus marginalis, Lethocerus indicus, Locusta migratoria, Gryllotalpa Gryllotalpa, Bombyx mori, Rhynchophorus ferrugineus, Blatta orientalis, Mesobuthus martensii, Gryllus bimaculatus, Acheta domesticus, Gryllus assimilis, Tenebrio molitor, Cicadidae, or any combination thereof.
39. The method of any one of aspects 30-38, wherein the multicellular marine organism is Arenicola marina, enchytraeus albidusm, Chironomidae, Hirudinea, Eisenia fetida, Perionyx excavates, Gastropoda, Mytilus edulis, Mytilus californianus, Crassostrea virginica, Crassostrea gigas, Stomolophus Meleagris, Lycastopsis catarractarum, Glycera, Nereidae, Artemia sp., Rotifera sp., Copepoda, Mercenaria mercenaria, Corbicula manilensis, Daphnia sp. Euphausiacea, Gammarus, or any combination thereof.
40. The method of any one of aspects 30-39, wherein the fungi is a yeast.
41. The method of aspect 40, wherein the yeast is Saccharomyces sp., Candida utilis, Lipomyces starkeyi and Phaffia rhodozyma, Fusarium moniliforme, Rhizopus niveus, Rhizopus oryzae, Aspergillus niger, Aspergillus oryzae, Candida guilliermondii, Candida lipolytica, Candida pseudotropicalis, Mucor pusillus Lindt, Mucor miehei, Rhizomucor miehei, Morteirella vinaceae, Endothia parasitica, Kluyveromyces lactis (previously called Saccharomyces lactis), Kluyveromyces marxianus, Lipomyces starkeyi, Rhodotorula colostri, Rhodotorula dairenensis, Rhodotorula glutinis, Rhodosporium diobovatum, Schizosaccharomyces pombe, Eremothecium ashbyii; Microfungi, optionally Fusarium venenatum, mushrooms, optionally Agaricus bisporus, Agaricus, Shiitake, Pleurotus ostreatus (oyster mushroom), Tremella fuciformis, Pleurotus eryngii, and/or Enoki, or any combination thereof.
42. A kit comprising the composition of any one of aspects 1-21.

1-42. (canceled)

43. A composition comprising:
an amount of an algae protein hydrolysate, an amount of a plant protein hydrolysate, an amount of a fungi protein hydrolysate, an amount of an insect protein hydrolysate, an amount of a multicellular marine organism protein hydrolysate, or any combination thereof.

44. The composition of claim 43, wherein the composition is a cell culture media supplement, a partially complete cell culture media, a complete cell culture media, or a serum replacer.

45. The composition of claim 43, wherein
(a) the algae protein hydrolysate is prepared from: a Chlorophyta (green algae), a Rhodophyta (red algae), a Stramenopiles (heterokonts), a Xanthophyceae (yellow-green algae), a Glaucocystophyceae (glaucocystophytes), a Chlorarachniophyceae (chlorarachniophytes), a Euglenida (euglenids), a Haptophyceae (coccolithophorids), a Chrysophyceae (golden algae), a Cryptophyta (cryptomonads), a Dinophyceae (dinoflagellates), a Haptophyceae (coccolithophorids), a Bacillariophyta (diatoms), a Eustigmatophyceae (eustigmatophytes), a Raphidophyceae (raphidophytes), a Scenedesmaceae (brown algae), a Phaeophyceae (brown algae), or any combination thereof;
(b) the plant protein hydrolysate is prepared from: Pea (Pisum sativum), Sorghum (Sorghum bicolor), rice (Oryza sativa), wheat (Triticum), Hemp (Cannabis sativa), quinoa (Chenopodium quinoa), soybean (Glycine max), Corn, beans family, Chickpeas, Lentils, Peanuts, Almonds, Chia seeds, Potatoes, Seitan, oat, Almond, barley, Brewers Spent Grain, or any combination thereof;
(c) the fungi protein hydrolysate is prepared from yeast;
(d) the insect protein hydrolysate is prepared from Hermetia illucens, Galleria mellonella, Acheta domesticus, Dytiscus marginalis, Lethocerus indicus, Locusta migratoria, Gryllotalpa Gryllotalpa, Bombyx mori, Rhynchophorus ferrugineus, Blatta orientalis, Mesobuthus martensii, Gryllus bimaculatus, Acheta domesticus, Gryllus assimilis, Tenebrio molitor, Cicadidae, or any combination thereof;
(e) the multicellular marine organism protein hydrolysate is prepared from Arenicola marina, enchytraeus albidusm, Chironomidae, Hirudinea, Eisenia fetida, Perionyx excavates, Gastropoda, Mytilus edulis, Mytilus californiams, Crassostrea virginica, Crassostrea gigas, Stomolophus Meleagris, Lycastopsis catarractarum, Glycera, Nereidae, Artemia sp., Rotifera sp., Copepoda, Mercenaria mercenaria, Corbicula manilensis, Daphnia sp. Euphausiacea, Gammarus, or any combination thereof; or
(f) any combination of (a)-(e).

46. The composition of claim 45, wherein the algae protein hydrolysate is prepared from:
(a) Chlamydomonas, Dunaliella, Haematococcus, Chlorella, Scenedesmaceae, and any combination thereof;
(b) Chlamydomonas reinhardtii, Dunaliella salina, Haematococcus pluvialis, Chlorella vulgaris, Acutodesmus obliquus, Scenedesmus dimorphus, or any combination thereof;
or
(c) a Chlorophyta comprising Chlamydomonas, Chlorella, or both, wherein the Chlamydomonas is Chlamydomonas reinhardtii and the Chlorella is Chlorella mimitissima or Chlorella sorokiniana.

47. The composition of claim 45, wherein the algae protein hydrolysate is prepared from a Rhodophyta and wherein the Rhodophyta
(a) is a Gigartinaceae or a Soliericeae; or
(b) comprises Chondrus crispus, Chondrus ocellatus, Eucheuma cottonii, Eucheuma spinosum, Gigartina acicularis, Gigartina pistillata, Gigartina radula, Gigartina stellate, Furcellaria fastigiata, Analipus japonicus, Eisenia bicyclis, Hizikia fusiforme, Kjellmaniella gyrata, Laminaria angustata, Laminaria longirruris, Laminaria Longissima, Laminaria ochotensis, Laminaria claustonia, Laminaria saccharina, Laminaria digitata, Laminaria japonica, Macrocystis pyrifera, Petalonia fascia, Scytosiphon lome, Gloiopeltis furcata, Porphyra crispata, Porhyra deutata, Porhyra perforata, Porhyra suborbiculata, Porphyra tenera, Rhodymenis palmate, or any combination thereof.

48. The composition of claim 45, wherein the yeast comprises Saccharomyces sp., Candida utilis, Lipomyces starkeyi and Phaffia rhodozyma, Fusarium moniliforme, Rhizopus niveus, Rhizopus oryzae, Aspergillus niger, Aspergillus oryzae, Candida guilliermondii, Candida lipolytica, Candida pseudotropicalis, Mucor pusillus Lindt, Mucor miehei, Rhizomucor miehei, Morteirella vinaceae, Endothia parasitica, Kluyveromyces lactis (previously called Saccharomyces lactis), Kluyveromyces marxiamis, Lipomyces starkeyi, Rhodotorula colostri, Rhodotorula dairenensis, Rhodotorula glutinis, Rhodosporium diobovatum, Schizosaccharomyces pombe, Eremothecium ashbyii; Microfungi, optionally Fusarium venenatum, mushrooms, optionally Agaricus bisporus, Agaricus, Shiitake, Pleurotus ostreatus (oyster mushroom), Tremella fuciformis, Pleurotus eryngii, and or Enoki, or any combination thereof.

49. The composition of claim 43, wherein
(a) the amount of each of the protein hydrolysates present in the composition ranges from any non-zero number greater than 0 to 100%;
(b) each hydrolysate present in the composition ranges in concentration as based on the total composition any non-zero concentration greater than 0 mg/mL to 100 mg/mL;
(c) the average peptide size of the composition ranges from 2-50 amino acids;
(d) the degree of hydrolysis for any one or more of the hydrolysates independently ranges from 0 to 100%;
(e) or any combination of (a)-(d).

50. The composition of claim 43, further comprising one or more nutrients, one or more pH indicators, one or more antibiotics, one or more antifungals, one or more biologic factors or agents, one or more buffering agents, one or more salts, one or more co-factors, one or more trace elements, a cell viability colorimetric agent, one or more cells, or any combination thereof.

51. The composition of claim 50, wherein the one or more cells are one or more eukaryotic cells.

52. A method of cell culture comprising:
culturing one or more cells in a composition according to claim 43.

53. The method of claim 52, wherein culturing further comprises, plating, growing, passaging, expanding, splitting, or any combination thereof, one or more times.

54. A method of preparing a composition of claim 43, the method comprising:
(a) blending one or more protein sources with water at a ratio of protein sources to water to form a slurry;
(b) adding alcalase to the slurry formed in (a) for a period of time to form a hydrolysate and one or more byproducts;
(c) inactivating enzymes present in the hydrolysate and the one or more byproducts; and
(d) separating the hydrolysate from the one or more byproducts, optionally by centrifugation.

55. The method of claim 54, further comprising storing and/or retaining at least the hydrolysate.

56. The method of claim 54, wherein step (b) is performed
(i) at about 60 degrees C.;
(ii) at a pH of about 6-8;
(iii) with agitation; or
(iv) any combination of (i)-(iv).

57. The method of claim 54, wherein (i) the one or more byproducts are oil, sludge, or both; (ii) (d) separating comprises centrifuging the hydrolysate and the one or more byproducts; or both (i) and (ii).

58. The method of claim 54, wherein the one or more protein sources are selected from a plant, a fungi, an algae, an insect, a multicellular marine organism, or any combination thereof.

59. The method of claim 54; wherein
(i) the plant is Pea (Pisum sativum), Sorghum (Sorghum bicolor), rice (Oryza sativa), wheat (Triticum), Hemp (Cannabis sativa), quinoa (Chenopodium quinoa), soybean (Glycine max), Corn, beans family, Chickpeas, Lentils, Peanuts, Almonds, Chia seeds, Potatoes, Seitan, Oat, Almond, Barley, Brewers Spent Grain, or any combination thereof;
(ii) the algae is Chlorophyta (green algae), Rhodophyta (red algae), Stramenopiles (heterokonts), Xanthophyceae (yellow-green algae), Glaucocystophyceae (glaucocystophytes), Chlorarachniophyceae (chlorarachniophytes), Euglenida (euglenids), Haptophyceae (coccolithophorids), Chrysophyceae (golden algae), Cryptophyta (cryptomonads), Dinophyceae (dinoflagellates), Haptophyceae (coccolithophorids), Bacillariophyta (diatoms), Eustigmatophyceae (eustigmatophytes), Raphidophyceae (raphidophytes), Scenedesmaceae and Phaeophyceae (brown algae), or any combination thereof;
(iii) the insect is Hermetia illucens, Galleria mellonella, Acheta domesticus, Dytiscus marginalis, Lethocerus indicus, Locusta migratoria, Gryllotalpa Gryllotalpa, Bombyx mori, Rhynchophorus ferrugineus, Blatta orientalis, Mesobuthus martensii, Gryllus bimaculatus, Acheta domesticus, Gryllus assimilis, Tenebrio molitor, Cicadidae, or any combination thereof;
(iv) the multicellular marine organism comprise a Arenicola marina, enchytraeus albidusm, Chironomidae, Hirudinea, Eisenia fetida, Perionyx excavates, Gastropoda, Mytilus edulis, Mytilus californianus, Crassostrea virginica, Crassostrea gigas, Stomolophus Meleagris, Lycastopsis catarractarum, Glycera, Nereidae, Artemia sp., Rotifera sp., Copepoda, Mercenaria mercenaria, Corbicula manilensis, Daphnia sp. Euphausiacea, Gammarus, or any combination thereof;
(v) the fungi comprises a yeast; or
(vi) any combination of (i)-(v).

60. The method of claim 59, wherein the algae is selected from
(i) Chlamydomonas, Dunaliella, Haematococcus, Chlorella, Scenedesmaceae, or any combination thereof; or
(ii) Chlamydomonas reinhardtii, Dunaliella salina, Haematococcus pluvialis, Chlorella vulgaris, Acutodesmus obliquus, Scenedesmus dimorphus, or any combination thereof;
or (iii) a Chlorophyta comprising Chlamydomonas, Chlorella, or both, wherein the Chlamydomonas is Chlamydomonas reinhardtii and the Chlorella is Chlorella minutissima or Chlorella sorokiniana.

61. The method of claim 59, wherein
(a) the Rhodophyta
(i) is a Gigartinaceae or a Soliericeae; or
(ii) comprises Chondrus crispus, Chondrus ocellatus, Eucheuma cottonii, Eucheuma spinosum, Gigartina acicularis, Gigartina pistillata, Gigartina radula, Gigartina stellate, Furcellaria fastigiata, Analipus japonicus, Eisenia bicyclis, Hizikia fusiforme, Kjellmaniella gyrata, Laminaria angustata, Laminaria longirruris, Laminaria Longissima, Laminaria ochotensis, Laminaria claustonia, Laminaria saccharina, Laminaria digitata, Laminaria japonica, Macrocystis pyrifera, Petalonia fascia, Scytosiphon lome, Gloiopeltis furcata, Porphyra crispata, Porhyra deutata, Porhyra perforata, Porhyra suborbiculata, Porphyra tenera, Rhodymenis palmate, or any combination thereof;
(b) the yeast comprises Saccharomyces sp., Candida utilis, Lipomyces starkeyi and Phaffia rhodozyma, Fusarium moniliforme, Rhizopus niveus, Rhizopus oryzae, Aspergillus niger, Aspergillus oryzae, Candida guilliermondii, Candida lipolytica, Candida pseudotropicalis, Mucor pusillus Lindt, Mucor miehei, Rhizomucor miehei, Morteirella vinaceae, Endothia parasitica, Kluyveromyces lactis (previously called Saccharomyces lactis), Kluyveromyces marxiams, Lipomyces starkeyi, Rhodotorula colostri, Rhodotorula dairenensis, Rhodotorula glutinis, Rhodosporium diobovatum, Schizosaccharomyces pombe, Eremothecium ashbyii; Microfungi, optionally Fusarium venenatum, mushrooms, optionally Agaricus bisporus, Agaricus, Shiitake, Pleurotus ostreatus (oyster mushroom), Tremella fuciformis, Pleurotus eryngii, and or Enoki, or any combination thereof; or
(c) both (a) and (b).

62. A kit comprising, the composition of claim 43.
